(19) United States
(12) Patent Application Publication
(10) Pub. No.: US 2024/0190965 A1
(43) Pub. Date: Jun. 13, 2024

(54) TARGETING ANTI-HUMAN PD 1H/VISTA TO TREAT HEMATOLOGIC DISORDERS

(71) Applicant: VANDERBILT UNIVERSITY, Nashville, TN (US)

(72) Inventor: Tae Kon Kim, Nashville, TN (US)

(21) Appl. No.: 18/531,168

(22) Filed: Dec. 6, 2023

Related U.S. Application Data
(60) Provisional application No. 63/386,707, filed on Dec. 9, 2022.

(51) Int. Cl. C07K 16/28; A61P 35/02

(52) U.S. Cl. CPC C07K 16/2818; A61P 35/02; C07K 16/2827

(57) ABSTRACT

As disclosed herein, (1) PD-1H is significantly up-regulated in human AML BM while PD-L1 expression is relatively low; (2) PD-1H is highly expressed on human AML blasts but not on normal CD34+ progenitors; (3) PD-1H expressed on AML blasts contributes to the induction of immune evasion in murine AML models; (4) genetic ablation or antibody blockade of PD-1H reverses immune evasion, leading to anti-leukemia effects in murine AML models and humanized AML models; (5) the effect of anti-PD-1H mAb could be maximized by blocking the PD pathway in murine AML models and humanized AML models. Therefore, disclosed herein is a method for treating a leukemia in a subject that involves co-administering to the subject a therapeutically effective amount of a checkpoint inhibitor and a therapeutically effective amount of an antibody that specifically binds PD-1H.

### CROSS-REFERENCE TO RELATED APPLICATIONS

This application claims benefit of U.S. Provisional Application No. 63/386,707, filed Dec. 9, 2022, which is hereby incorporated herein by reference in its entirety.

### STATEMENT OF GOVERNMENT INTEREST

This invention was made with Government Support under Grant No. CA016359 awarded by the National Institutes of Health. The Government has certain rights in the invention.

### BACKGROUND OF THE INVENTION

Acute myeloid leukemia (AML) is a heterogenous clonal disorder that is characterized by uncontrolled clonal expansion of myeloid progenitor cells (blasts) that leads to bone marrow (BM) failure (Döhner H, et al. N Engl J Med 2015 373(12): 1136-1152). AML is the most common acute leukemia in adults(1). The incidence of AML is 20,240 per year, and over 11,400 patients die annually from this disease in the United States. Despite progress in our understanding of the pathology and genetics of this disease (Winer E S, et al. Ther Adv Hematol 2019 10), as well as extensive development of targeted therapeutic modalities (Cortes J E et al. et al. Leukemia 2019 33(2):379-389; Daver N et al. Blood Cancer J. 2020 10(10): 1-12; DiNardo C D et al. New England Journal of Medicine Jun. 2, 2018; DiNardo C D et al. N Engl J Med 2020 383(7):617-629; Krauss A C et al. Clin Cancer Res 2019 25(9):2685-2690; Lambert J et al. Haematologica 2019 104(1):113-119; Lancet J E et al. JCO 2018 36(26):2684-2692; Norsworthy K J et al. The Oncologist 2018 23(9): 1103-1108; Norsworthy K J et al. Clin Cancer Res 2019 25(20):6021-6025; Norsworthy K J et al. Clin Cancer Res 2019 25(11):3205-3209; Perl A E et al. New England Journal of Medicine 2019 381(18): 1728-1740; Stein E M et al. Blood 2017 130(6):722-731; Stone R M et al. New England Journal of Medicine 2017 377(5):454-464), the mainstay for AML treatment has remained the combination of anthracycline/cytarabine, which was developed in the 1970s (Lichtman M A. Blood Cells Mol. Dis. 2013 50(2): 119-130).

Recently, the antibody therapy targeting the PD-1/B7-H1 (PD-L1) pathway (collectively called anti-PD therapy) has been at the forefront of cancer therapy (Dong H, et al. Nat. Med. 1999 5(12): 1365-1369; Dong H et al. Nat. Med. 2002 8(8): 793-800; Kim T K, et al. Trends Immunol. 2018 39(8): 624-631; Topalian S L et al. New England Journal of Medicine 2012 366(26): 2443-2454). Anti-PD therapy was developed based on early findings showing selective upregulation of B7-H1 in the tumor microenvironment (TME) by IFN-g, leading to dysfunction of tumor infiltrating T lymphocytes (TILs) upon its engagement of PD-1, a mechanism called adaptive immune resistance (Dong H et al. Nat. Med. 2002 8(8):793-800; Kim T K, et al. Trends Immunol. 2018 39(8):624-631; Chen L, et al. J Clin Invest 2015 125(9):3384-3391; Sanmamed M F, et al. Cell 2018 175(2):313-326). Currently, anti-PD therapy has been approved by the U.S. Food and Drug Administration (FDA) for the treatment of more than 25 indications in common cancers including solid tumors and hematopoietic malignancies (Ansell S M et al. New England Journal of Medicine 2015 372(4):311-319; Ferris R L et al. New England Journal of Medicine 2016 375(19): 1856-1867; Herbst R S et al. Nature 2014 515(7528):563-567; Horn L et al. New England Journal of Medicine 2018 379(23):2220-2229). Despite these exciting developments, clinical efficacy of anti-PD therapy in AML remains obscure. Single-agent anti-PD-1 or anti-PDL1 monoclonal antibody (mAb) trials in AML has shown marginal response rates (Daver N et al. Cancer Discov 2019 9(3):370-383; Garcia-Manero G et al. Blood 2016; 128(22):344-344; Vandsemb E N, et al. Cancer 2019 125(9): 1410-1413; Williams P et al. Cancer 2019 125(9): 1470-1481; Zeidner J F et al. Curr Drug Targets 2017 18(3):304-314). The marginal response to anti-PD therapy in AML indicates that different mechanisms of immune evasion other than the PD pathway may be present.

### SUMMARY OF THE INVENTION

Programmed Death-1 Homolog (PD-1H, also known as V domain immunoglobulin suppressor of T cell activation (VISTA), V-set immunoregulatory receptor (VSIR), C10orf54, DD1a and Gi24) is a co-inhibitory molecule of the immunoglobulin superfamily and is broadly found in hematopoietic cells (Flies D B, et al. J. Immunol. 2011 187(4): 1537-1541; Wang L et al. J. Exp. Med. 2011 208(3):577-592). PD-1H delivers an inhibitory signal as a ligand to T cells (Wang L et al. J. Exp. Med. 2011 208(3):577-592; Lines J L et al. Cancer Res 2014 74(7): 1924-1932), yet PD-1H on T cells also receives inhibitory signals as a receptor (Flies D B, et al. J. Immunol. 2011 187(4): 1537-1541; EITanbouly M A et al. Science 2020 367(6475): eaay0524; Flies D B, et al. J. Immunol. 2015 194(11):5294-5304; Han X et al. Science Translational Medicine 2019 11(522); Flies D B et al. J. Clin. Invest. 2014 124(5): 1966-1975). Several counter-receptors of PD-1H have been identified, but their immunological functions remain to be elucidated (Johnston R J et al. Nature 2019 574(7779):565-570; Mehta N et al. Cell Rep 2019 28(10):2509-2516.e5; Wang J et al. Immunology 2019 156(1):74-85). PD-1H is expressed mainly in hematopoietic cells including T cells, monocytes, macrophages, and dendritic cells (Flies D B, et al. J. Immunol. 2011 187(4): 1537-1541; Lines J L et al. Cancer Res 2014 74(7): 1924-1932). The presence of PD-1H in normal tissues/cells support its function as a homeostatic regulator including maintenance of CD4+ T cells in quiescence (EITanbouly M A et al. Science 2020 367(6475): eaay0524). In preclinical murine models, PD-1H has been shown to induce immune evasion, and genetic ablation or antibody blockade of PD-1H promotes T cell-mediated immunity and suppresses tumor growth (Wang L et al. J. Exp. Med. 2011 208(3):577-592; Johnston R J et al. Nature 2019 574(7779):565-570; Flies D B et al. J Clin Invest 2014 124(5): 1966-1975; Le Mercier I et al. Cancer Res. 2014 74(7): 1933-1944; Xu W et al. Cancer Immunol Res 2019 7(9): 1497-1510).

As disclosed herein, (1) PD-1H is significantly up-regulated in human AML BM while PD-L1 expression is relatively low; (2) PD-1H is highly expressed on human AML blasts but not on normal CD34+ progenitors; (3) PD-1H expressed on AML blasts contributes to the induction of immune evasion in murine AML models; (4) genetic ablation or antibody blockade of PD-1H reverses immune evasion, leading to anti-leukemia effects in murine AML models and humanized AML models; (5) the effect of anti-PD-1H mAb could be maximized by blocking the PD pathway in murine AML models and humanized AML models.

Disclosed herein is a method for treating a leukemia in a subject that involves co-administering to the subject a therapeutically effective amount of a checkpoint inhibitor and a therapeutically effective amount of an antibody that specifically binds PD-1H.

In some embodiments, the leukemia is an acute myeloid leukemia (AML). In some embodiments, the leukemia is a myelodysplastic syndrome (MDS). In some embodiments, the leukemia is a Philadelphia chromosome-positive acute lymphoblastic leukemia (Ph+ ALL).

In some embodiments, the checkpoint inhibitor comprises an anti-PD-1 antibody, anti-PD-L1 antibody, anti-CTLA-4 antibody, or a combination thereof.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIGS. 1A to 1G show PD-1H protein is highly expressed on AML blasts. FIG. 1A shows immunohistochemical staining of human PD-1H and PD-L1 in AML. Validation of PD-1H and PD-L1 staining in human placenta in left panels. IHC staining of PD-1H and PD-L1 in human AML BM core biopsies in the right panels (representative picture, monocytic AML). 400× magnification. Scale bars=20 μm. FIG. 1B shows pathologic score of PD-1H and PD-L1 expression in AML BM core biopsies. Scores of 0, 1, 2, and 3 indicate that <5%, 5-20%, 20-40%, and >40% of of AML blasts, respectively, showed PD-1H or PD-L1 expression. FIG. 1C shows flow cytometric analysis of healthy donor (HD) CD34+ cells (far left), AML blasts (either CD34+ or CD33+) (second panel), HD CD11b+ myeloid cells (third panel), and HD CD3+ T cells (far right). FIG. 1D shows change in (A) mean fluorescence intensity (MFI) (MFI in PD-1H staining—MFI in isotype staining). Mean value of ΔMFI in HD CD34+ progenitors vs, mean value of ΔMFI in AML CD34+ blasts=76±26.8 (N=5) vs. 11,469±4,873 (N=26), p=0.02. P value determined by Student's T test. Error bars represent standard error of mean (SEM). FIG. 1E shows flow cytometric analysis of AML subsets (t(8;21), complex karyotype, non-monocytic, and monocytic (left to right). FIG. 1F shows mean value of ΔMFI in t(8;21) vs. in monosomic complex karyotype AML (551±145 (N=4) vs. 9,469±3,880 (N=8), P value determined by one way ANOVA. Error bars represent SEM. Ns: not significant, *P<0.05. FIG. 1G shows mean value of ΔMFI in non-monocytic vs. monocytic AML (822±155 (N=19) vs. 23,881±9,533 (N=7)). P value determined by one way ANOVA. Error bars represent SEM. * P<0.05.

FIGS. 2A to 2F show AML surface PD-1H inhibits T cell infiltration, leading to immune evasion. FIG. 2A shows syngeneic mouse leukemia model using tail vein injection with myeloid leukemia cells (C1498). Mouse leukemia cells expressing PD-1H (C1498FF-PD-1H) or cells not expressing PD-1H (C1498FF-mock) were transplanted into C57BL/6 mice (B6 mice) and assessed for in vivo leukemia proliferation using bioluminescence. FIG. 2B shows in vivo proliferation of C1498FF-mock vs. C1498FF-PD-1H cells in B6 WT mice (N=7). Radiance indicates the mean value per group and error bars represent SEM, NS: not significant. P value determined by Student's T test at each timepoint ***P<0.001, *P<0.05. These experiments were repeated three times. Repeated measures were determined by ANOVA with two factors (P>0.05, no difference among experiments). FIG. 2C shows in vivo proliferation of C1498FF-mock vs. C1498FF-PD-1H cells in NSG mice (N=3) (representative images on day 21 on the right side). Radiance indicates the mean value per group and error bars represent SEM. P value determined by Student's T test at each timepoint. NS: not significant. Repeated measures were determined by ANOVA with two factors (P>0.05, no difference among experiments). FIG. 2D shows in vitro growth of C1498FF-PD-1H tumors compared with C1498FF-mock tumors. Statistical analysis was done using student T test. NS: not significant. FIG. 2E shows syngeneic mouse model using s.c. injection with C1498 cells. C1498FF-PD-1H cells or C1498FF-mock cells were s.c. injected into the flanks of B6 mice and the tumor volume was assessed. Mean tumor volume±SED. P value determined by Student's T test at each timepoint. N=5 per group, P=0.07. Mice were sacrificed on day 12 and tumor tissues were removed for mass cytometry assay. FIG. 2F shows quantification of immune subsets in mass cytometry data in C1498FF-PD-1H tumors compared with C1498FF-mock tumors (N=5 per group, P value determined by Student's T test. Error bars represent SEM. **p<0.01, *p<0.05).

FIGS. 3A to 3D show host-derived PD-1H induces immune evasion in AML. FIG. 3A shows syngeneic mouse leukemia model using tail vein injection with myeloid leukemia cells (C1498). Mouse leukemia cells (C1498FF-mock) were transplanted into B6 PD-1H WT or PD-1H KO mice or lineage-specific PD-1H KO mice. In vivo proliferation was assessed by bioluminescence. FIG. 3B shows in vivo anti-leukemia effect of genetic deletion of PD-1H in host mice. Radiance indicates the mean value per group and error bars represent SEM. P value determined by Student's T test at each timepoint. N=5 per group, p=0.04. These experiments were repeated three times. Repeated measures were determined by ANOVA with two factors (P>0.05, no difference among experiments). FIG. 3C shows in vivo anti-leukemia effect of myeloid lineage-specific deletion of PD-1H in host mice. Bioluminescence was compared in LysM (lysozyme)-Cre+PD-1H-floxed mice with control-PD-1H-floxed mice. Radiance indicates the mean value per group and error bars represent SEM. P value determined by Student's T test at each timepoint. Error bars represent SEM. *P<0.05. N=9 per group. Representative data was combined from two independent experiments. Repeated measures were determined by ANOVA (P>0.05, no difference among experiments). FIG. 3D shows in vivo anti-leukemia effect of T cell lineage-specific deletion of PD-1H in host mice. Bioluminescence was compared in Lck-Cre+PD-1H-floxed mice vs. control-PD-1H-floxed mice. Radiance indicates the mean value per group and error bars represent SEM. P value determined by Student's T test at each timepoint. Error bars represent SEM. N=6 per group, NS: not significant. Repeated measures were determined by ANOVA (P>0.05, no difference among experiments).

FIGS. 4A to 4G show anti-mouse PD-1H mAb reverses immune evasion induced by mouse AML surface PD-1H. FIG. 4A shows PD-1H suppressed T cell activation. Inhibition of OT-1 T cells by mouse PD-1H on 293-KbOVA cells. T cell proliferation was assessed by carboxyfluorescein succinimidyl ester (CFSE) dilution. The diluted population was assessed by the percentage of total T cells. FIGS. 4B and 4C show B6 PD-1H KO mice transplanted with myeloid leukemia cells expressing full length PD-1H (C1498FF-PD-1H FL) and treated with anti-mPD-1H mAb (13F3) (FIG. 4B). Mice were assessed for in vivo leukemia proliferation using bioluminescence (FIG. 4C). A total of 200 μg of 13F3 or isotype control mAb was intraperitoneally injected every 4 days from day 1 of transplantation of C1498FF-PD-1H cells (total 4 doses). Radiance indicates the mean value per group and error bars represent SEM. P value determined by Student's T test at each timepoint. N=5, *p<0.05, ***p<0.01. NS: not significant. FIGS. 4D to 4F show in vivo growth of C1498FF-PD-1H s.c. tumor in B6 WT mice following anti-mPD-1H mAb treatment. A total of 200 μg of 13F3 or isotype control mAb was intraperitoneally injected every 4 days from day 0 after s.c. injection of C1498FF-PD-1H cells (total 3 doses). FIG. 4D shows tumor size was significantly smaller in the 13F3 treatment group compared with the isotype treatment group. Mean tumor volume±SED. Error bars represent SEM. N=6 per group, *p<0.05. FIGS. 4E and 4F show C1498FF-PD-1H s.c. tumor growth with 13F3 or isotype mAb treatment in B6 WT mice depleted of T cells or NK cells. N=6, *p<0.05, ***p<0.01. NS: not significant. P value determined by Student's T test at each timepoint. FIG. 4G shows immune cell subsets infiltrated in C1498FF-PD-1H tumors were assessed using mass cytometry. Left: percentages of Granzyme B+CD8+ T cells in total CD8+ T cells. Right: percentages of effector memory phenotype (CD44+CD62L−) CD8+ T cells in total CD8+ T cells. P value determined by Student's T test. Error bars represent SEM. *p<0.05; **p<0.01.

FIGS. 5A to 5E shows anti-human PD-1H mAb reverses immune evasion induced by human AML surface PD-1H. FIG. 5A shows PD-1H suppressed T cell activation. Inhibition of polyclonal human T cells by human PD-1H on AML (HL-60). T cell proliferation was assessed by CFSE dilution. The diluted population was assessed by the percentage of total T cells. FIG. 5B shows the role of human AML PD-1H using a humanized mouse model. Human myeloid leukemia cells expressing PD-1H (HL-60-PD-1H) or not expressing PD-1H (HL-60-mock) were s.c. injected into NSG or NSG-S mice reconstituted with human peripheral blood mononuclear cells. Mice were sacrificed on day 14 and tumor tissues were removed to assess the size and to carry out IHC. FIGS. 5C and 5D show the volume of excised leukemia tumors (HL-60 (left), MOLM14 (middle), THP1 (far right)) expressing PD-1H or not expressing PD-1H or PD-1H-expressing leukemia tumors following anti-hPD-1H mAb treatment (N=5 per group, *p<0.05). P value determined by One way ANOVA (FIG. 5C), and Student's T test (FIG. 5D). Mean tumor volume±SED. Error bars represent SEM. Picture depicts HL-60 tumors removed from humanized NSG-S mice. FIG. 5E shows immunohistochemistry of leukemia tumors expressing PD-1H following anti-hPD-1H mAb to assess CD4+ or CD8+ T cell infiltration (HL-60) and CD3 (MOLM14).

FIGS. 6A to 6C show mouse PD-1H blockade confers a synergistic anti-leukemic effect with mouse PD-1 blockade. FIG. 6A shows syngeneic mouse leukemia model using tail vein injection with mouse myeloid leukemia cells expressing PD-1H (C1498FF-PD-1H) were transplanted into C57BL/6 mice (B6 mice) and then treated with anti-PD-1 and/or anti-PD-1H mAb. Syngeneic mouse leukemia model using tail vein injection with mouse myeloid leukemia cells not expressing PD-1H (C1498FFmock) were transplanted into WT B6 mice or PD-1H KO mice and assessed for in vivo anti-leukemia effect of genetic deletion of PD-1H in host mice with or without anti-PD-1 mAb. FIG. 6B shows synergistic anti-leukemia effect of anti-PD-1 mAb with anti-PD-1H mAb. In vivo proliferation was assessed by bioluminescence (left) and survival by a Kaplan-Meier plot (right). Radiance indicates the mean value per group and error bars represent SEM. Data from two experiments were combined (N=10). FIG. 6C shows synergistic anti-leukemia effect of genetic deletion of PD-1H in host mice (PD-1H KO) with anti-PD-1 mAb. In vivo proliferation was assessed by bioluminescence (left) and survival by a Kaplan-Meier plot (right). Radiance indicates the mean value per group and error bars represent SEM. Data from two experiments were combined (N=10). FIG. 6B, 6C: P value determined by simple linear regression method for statistical analysis of radiance and Log-rank test for survival. * P<0.05, **P<0.01, ***P<0.001, ****P<0.0001. These experiments were repeated two times. Repeated measures were determined by ANOVA with two factors (P>0.05, no difference among experiments).

FIGS. 7A and 7B show human PD-1H blockade confers a synergistic anti-leukemic effect with human PD-1 blockade. A humanized AML mouse model to demonstrate a synergistic anti-leukemia effect of anti-hPD-1 with anti-hPD-1H mAb. Human myeloid leukemia cells expressing PD-1H (THP1-WT) or not expressing PD-1H (THP1-PD-1H KO) were s.c. injected into NSG mice reconstituted with human peripheral blood mononuclear cells. Tumor volume was assessed on day 2, 6, 9. AntihPD-1 (100 ug) and/or anti-hPD-1H mAb (100 ug) were injected on day 7. Day 9 tumor volume was represented. Mean tumor volume±SED. Error bars represent SEM. N=5. NS: not significant, *P<0.05, ***P<0.001, ****P<0.001. P value determined by One way ANOVA.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 1011 M⁻¹, and 1012 M⁻¹ or more) with that second molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### Anti-PD-1H Antibodies

The ability of Abs to cross-compete for binding to an antigen indicates that these Abs bind to the same epitope region (i.e., the same or an overlapping epitope) of the antigen and sterically hinder the binding of other cross-competing Abs to that particular epitope region.

An anti-PD-1H Ab of the invention further can be prepared using an Ab having one or more of the VH and/or VL sequences disclosed herein as starting material to engineer a modified Ab, which modified Ab may have altered properties from the starting Ab. An Ab can be engineered by modifying one or more residues within one or both variable regions (i.e., VH and/or VL), for example within one or more CDR regions and/or within one or more framework regions. Additionally or alternatively, an Ab can be engineered by modifying residues within the constant region(s), for example, to alter the effector function(s) of the Ab. Specific modifications to Abs include CDR grafting, site-specific mutation of amino acid residues within the VH and/or V_{K} CDR1, CDR2 and/or CDR3 regions to thereby improve one or more binding properties (e.g., affinity) of the Ab, site-specific mutation of amino acid residues within the VH and/or V_{K} framework regions to decrease the immunogenicity of the Ab, modifications within the Fe region, typically to alter one or more functional properties of the Ab, such as serum half-life, complement fixation, Fc receptor binding, and/or antigen-dependent cellular cytotoxicity, and chemical modification such as pegylation or alteration in glycosylation patterns to increase or decrease the biological (e.g., serum) half life of the Ab.

Anti-PD-1H Abs of the invention also include antigen-binding portions of the above Abs. It has been amply demonstrated that the antigen-binding function of an Ab can be performed by fragments of a full-length Ab. Examples of binding fragments encompassed within the term “antigen-binding portion” of an Ab include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; and (iv) a Fv fragment consisting of the VL and VH domains of a single arm of an Ab.

These fragments, obtained initially through proteolysis with enzymes such as papain and pepsin, have been subsequently engineered into monovalent and multivalent antigen-binding fragments. For example, although the two domains of the Fv fragment, VL and VH, are coded for by separate genes, they can be joined, using recombinant methods, by a synthetic linker peptide that enables them to be made as a single protein chain in which the VL and VH regions pair to form monovalent molecules known as single chain variable fragments (scFv). Divalent or bivalent scFvs (di-scFvs or bi-scFvs) can be engineered by linking two scFvs in within a single peptide chain known as a tandem scFv which contains two VH and two VL regions. ScFv dimers and higher multimers can also be created using linker peptides of fewer than 10 amino acids that are too short for the two variable regions to fold together, which forces the scFvs to dimerize and produce diabodies or form other multimers. Diabodies have been shown to bind to their cognate antigen with much higher affinity than the corresponding scFvs, having dissociation constants up to 40-fold lower than the KD values for the scFvs. Very short linkers (≤3 amino acids) lead to the formation of trivalent triabodies or tetravalent tetrabodies that exhibit even higher affinities for to their antigens than diabodies. Other variants include minibodies, which are scFv-CH3 dimers, and larger scFv-Fc fragments (scFv-CH2-CH3 dimers), and even an isolated CDR may exhibit antigen-binding function. These Ab fragments are engineered using conventional recombinant techniques known to those of skill in the art, and the fragments are screened for utility in the same manner as are intact Abs. All of the above proteolytic and engineered fragments of Abs and related variants (see Hollinger et al., 2005; Olafsen et al., 2010, for further details) are intended to be encompassed within the term “antigen-binding portion” of an Ab.

### Checkpoint Inhibitors

The disclosed CARs can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

### Anti-PD-1 Antibodies

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

### Anti-PD-L1 Antibodies

Each of the anti-PD-L1 HuMAbs disclosed in U.S. Pat. No. 7,943,743 has been demonstrated to exhibit one or more of the following characteristics (a) binds to human PD-L1 with a KD of 1×10⁻⁷ M or less; (b) increases T-cell proliferation in a Mixed Lymphocyte Reaction (MLR) assay; (c) increase interferon-γ production in an MLR assay; (d) increase IL-2 secretion in an MLR assay; (e) stimulates Ab responses; (f) inhibits the binding of PD-L1 to PD-1; and (g) reverses the suppressive effect of T regulatory cells on T cell effector cells and/or dendritic cells. Anti-PD-L1 Abs include mAbs that bind specifically to human PD-L1 and exhibit at least one, preferably at least four, of the preceding characteristics. U.S. Pat. No. 7,943,743 exemplifies ten anti-PD-1 HuMAbs: 3G10, 12A4 (also referred to herein as BMS-936559), 10A5, 5F8, 10H10, 1B12, 7H1, 11E6, 12B7, and 13G4, which is incorporated by reference for these antibodies.

### Pharmaceutical Compositions

Antibodies disclosed herein may be constituted in a composition, e.g., a pharmaceutical composition, containing one Ab or a combination of Abs, or an antigen-binding portion(s) thereof, and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). A pharmaceutical composition of the invention may include one or more pharmaceutically acceptable salts, anti-oxidant, aqueous and nonaqueous carriers, and/or adjuvants such as preservatives, wetting agents, emulsifying agents and dispersing agents.

Dosage regimens are adjusted to provide the optimum desired response, e.g., a therapeutic response or minimal adverse effects. The dosage ranges from about 0.0001 to about 100 mg/kg, usually from about 0.001 to about 20 mg/kg, and more usually from about 0.01 to about 10 mg/kg, of the subject's body weight. Preferably, the dosage is within the range of 0.1-10 mg/kg body weight. For example, dosages can be 0.1, 0.3, 1, 3, 5 or 10 mg/kg body weight, and more preferably, 0.3, 1, 3, or 10 mg/kg body weight. The dosing schedule is typically designed to achieve exposures that result in sustained receptor occupancy (RO) based on typical pharmacokinetic properties of an Ab. An exemplary treatment regime entails administration once per week, once every two weeks, once every three weeks, once every four weeks, once a month, once every 3 months or once every three to 6 months. The dosage and scheduling may change during a course of treatment. For example, dosing schedule may comprise administering the Ab: (i) every two weeks in 6-week cycles; (ii) every four weeks for six dosages, then every three months; (iii) every three weeks; (iv) 3-10 mg/kg body weight once followed by 1 mg/kg body weight every 2-3 weeks. Considering that an IgG4 Ab typically has a half-life of 2-3 weeks, a preferred dosage regimen for the disclosed antibodies comprises 0.3-10 mg/kg body weight, preferably 3-10 mg/kg body weight, more preferably 3 mg/kg body weight via intravenous administration, with the Ab being given every 14 days in up to 6-week or 12-week cycles until complete response or confirmed progressive disease.

In some methods, two or more mAbs with different binding specificities are administered simultaneously, in which case the dosage of each Ab administered falls within the ranges indicated. Antibody is usually administered on multiple occasions. Intervals between single dosages can be, for example, weekly, every 2 weeks, every 3 weeks, monthly, every three months or yearly. Intervals can also be irregular as indicated by measuring blood levels of Ab to the target antigen in the patient. In some methods, dosage is adjusted to achieve a plasma Ab concentration of about 1-1000 μg/ml and in some methods about 25-300 μg/ml.

Alternatively, the Ab can be administered as a sustained release formulation, in which case less frequent administration is required. Dosage and frequency vary depending on the half-life of the Ab in the patient. In general, human Abs show the longest half-life, followed by humanized Abs, chimeric Abs, and nonhuman Abs. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present invention can be administered via one or more routes of administration using one or more of a variety of methods well known in the art. As will be appreciated by the skilled artisan, the route and/or mode of administration will vary depending upon the desired results.

### Therapeutic Methods

Disclosed herein is a method for immunotherapy of a subject afflicted with cancer, which method comprises administering to the subject a composition comprising a therapeutically effective amount of an antibody or an antigen-binding portion thereof that disrupts the interaction of PD-1H with its ligand alone or in combination with another immunotherapy, such as antibody or an antigen-binding portion thereof that disrupts the interaction of PD-1 with PD-L1 and/or PD-L2.

The disclosed immunotherapies can be used in combination with other cancer therapies. In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent is a targeted agent, such as ibrutinib or idelalisib.

In some embodiments, such an additional therapeutic agent is an epigenetic modifier such as azacitdine or vidaza.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbB1 (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with a CARs for treating the disorders as described above may be an anti-cancer cytokine, chemokine, or combination thereof. Examples of suitable cytokines and growth factors include IFNγ, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C—C chemokine families. Suitable cytokines include cytokine derivatives, cytokine variants, cytokine fragments, and cytokine fusion proteins.

In some embodiments, a therapeutic agent may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed immunotherapies are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: PD-1H/VISTA Mediates Immune Evasion in Acute Myeloid Leukemia

### Results

VSIR (PD-1H) mRNA is Highly Upregulated in AML and Correlated with Poor Survival

We and others have previously reported that PD-1H is broadly expressed on mouse normal hematopoietic cells, including myeloid immune cells and T cells (Flies D B, et al. J Immunol. 2011 187(4): 1537-1541; Wang L, et al. J Exp Med. 2011 208(3):577-592; Lines J L, et al. Cancer Res. 2014 74(7): 1924-1932). PD-1H was also reported to be expressed in some human solid tumor tissues including prostate cancer (Gao J, et al. Nat Med. 2017 23(5):551-555), pancreatic cancers (Blando J, et al. Proc Natl Acad Sci USA. 2019 116(5): 1692-1697; Liu J, et al. Pancreas. 2018 47(6):725-731), and melanoma (Blando J, et al. Proc Natl Acad Sci USA. 2019 116(5): 1692-1697; Kuklinski L F, et al. Cancer Immunol Immunother. 2018 67(7): 1113-1121; Rosenbaum S R, et al. Cell Rep. 2020 30(2):510-524.e6), mostly in tumor-infiltrating immune cells. By analyzing the Cancer Genome Atlas (TCGA) database, we found that expression of VSIR (PD-1H) mRNA in AML is the highest among over 30 different human cancer types (The Cancer Genome Atlas Research Network, 2013). In addition, VSIR is one of co-inhibitory molecules that are expressed higher than others in AML. We next determined VSIR expression among AML subgroups based on the French-American-British classification of AML using TCGA. Interestingly, M4 (myelomonocytic) and M5 (monocytic) AML revealed the highest expression of VSIR among AML subsets. These findings are consistent with preferential expression of VSIR on normal myeloid cells.

We next investigated whether VSIR expression is associated with cytogenetic and molecular aberrations that determine the prognosis of AML (Bloomfield C D, et al. Cancer Res. 1998 58(18):4173-4179; Grimwade D, et al. Blood. 1998 92(7):2322-2333; Keating M J, et al. Leukemia. 1988 2(7):403-412). For instance, AML harboring RUNX1-RUNX1T1 (t(8;21)), PML-RARa (t(15;17)) or inv (16) is associated with more favorable prognosis than AML with a complex or monosomal karyotype. VSIR expression was significantly lower in favorable risk AML (i.e., RUNX1-RUNX1T1 (t(8;21)), PML-RARa (t(15;17)) than in intermediate and poor risk AML (i.e. intermediate risk: NPM1 mutation, normal karyotype etc.; poor risk: complex karyotype, monosomy (del (5), del (7)) etc.). However, in some good risk AML types, such as CBFB-MYH11 (inv (16), t(16;16)) which is often associated with monocytic differentiation, PD-1H had expression levels comparable to intermediate and poor risk AML. Although these significant differences in VSIR expression were evident based on cytogenetics in AML, molecular mutations including DNMT3A, 11q23 amplification, FLT3, NPM1 and TP53 did not correlate significantly with VSIR RNA levels. Therefore, decreased expression of VSIR is associated with particular cytogenetic aberrations such as t(8;21) and t(15;17) in AML.

Survival analyses in TCGA to compare the VSIR^{high} quartile AML population with the VSIR^{low} quartile AML population showed that the VSIR^{low} AML population survived longer than the VSIR^{high} AML population. Collectively, our findings suggest a potential role of PD-1H up-regulation in immune evasion in AML.

### PD-1H is Highly Expressed on the Surface of Human AML Blasts

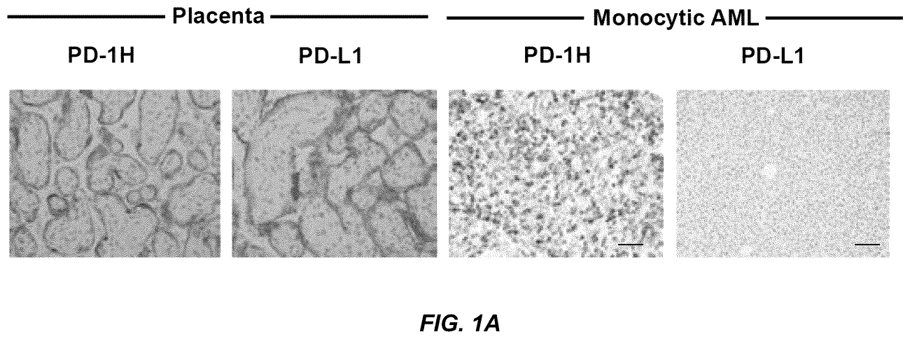

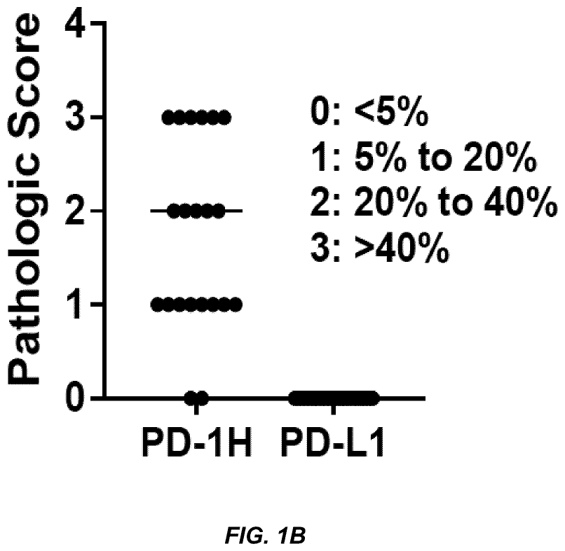

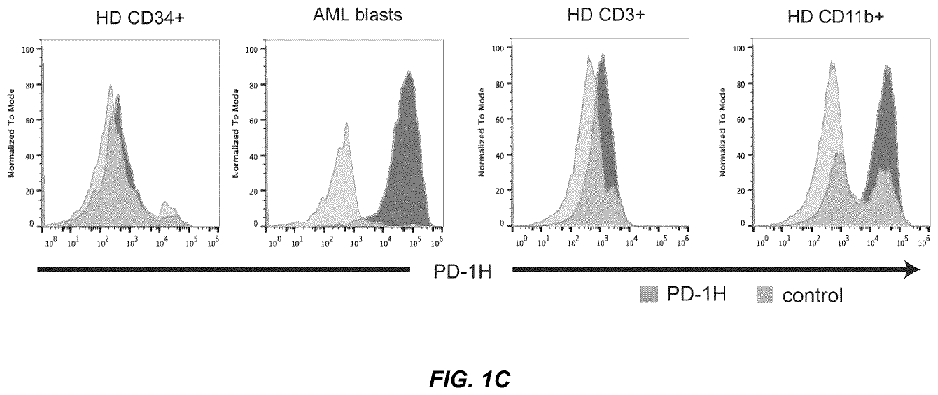

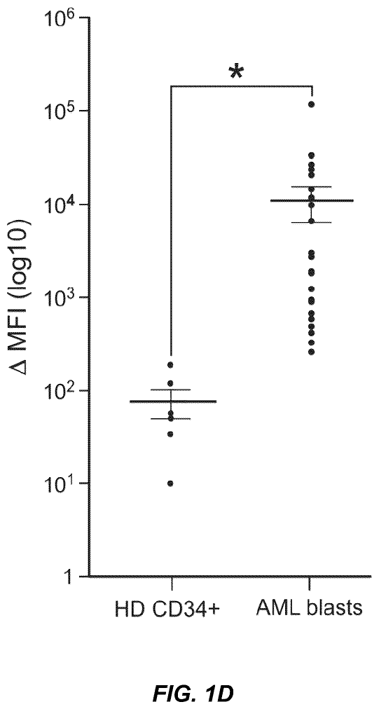

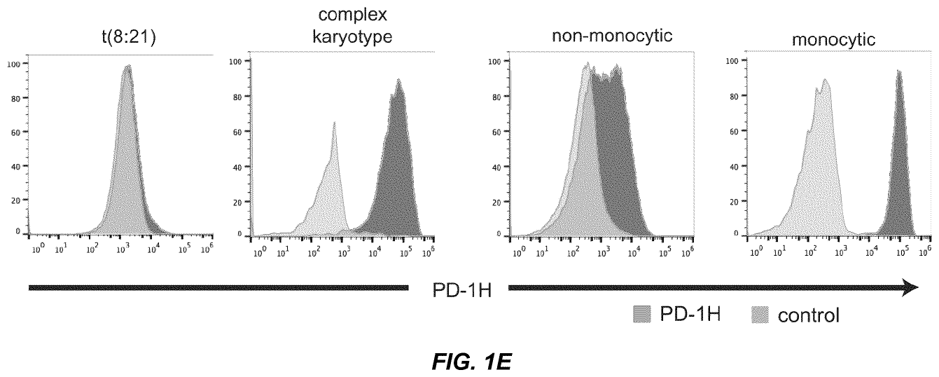

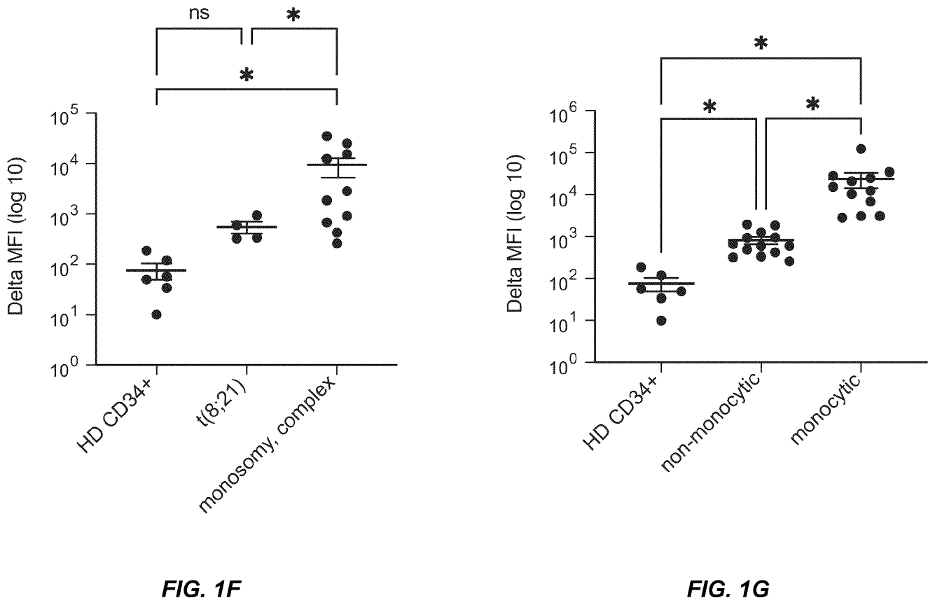

To determine the expression of PD-1H surface protein in human AML, we evaluated BM core biopsies sampled from 21 AML patients by immunohistochemistry (IHC) (Table 1A). Interestingly, PD-1H surface protein was expressed on AML blasts in BM from 19 out of 21 AML patients (higher than IHC score 1: >5% of blasts) (FIGS. 1A & 1B, Table 1). PD-L1 expression, however, was largely minimal on AML blasts (FIGS. 1A & 1B), although we saw weak expression in normal myeloid subsets. Our data is somewhat contradictory to previous reports that demonstrated PD-L1 expression in myeloid leukemia (Zajac M, et al. Br J Haematol. 2018 183(5):822-825; Yang H, et al. Leukemia. 2014 28(6): 1280-1288). But of note, these prior data was based on mRNA expression of PD-L1 compared with our assay to detect PD-L1 protein. These data suggest that PD-1H may be one of the important immune modulators in AML. Among subtypes of AML, complex karyotype AML had higher cell surface expression of PD-1H than t(8;21) and t(15;17) AML, suggesting poor risk AML such as complex karyotype tends toward higher expression of PD-1H than favorable risk AML such as t(8;21) and t(15;17). More obviously, PD-1H expression was significantly higher in monocytic AML than non-monocytic AML. These data are consistent with TCGA mRNA expression data.

We confirmed that PD-1H cell surface staining in IHC analysis is specific by flow cytometry based on positive control (HL-60-PD-1H), negative control (HL-60-mock) and isotype control staining. The specificity of PD-1H staining was also validated using several different clones of anti-human PD-1H (hPD-1H) mAb and different staining protocols (e.g., fixation or non-fixation prior to staining). Among three anti-hPD-1H mAbs, one clone, MIH65, provided specific staining before or after fixing cells that allowed us to use this mAb with either fresh, cryopreserved, non-fixed, or fixed AML BM cells for flow cytometric analyses. Consistent with prior reports (Flies D B, et al. J Immunol. 2011 187(4): 1537-1541; Lines J L, et al. Cancer Res. 2014 74(7): 1924-1932; Le Mercier I, et al. Cancer Res. 2014 74(7): 1933-1944), the flow cytometry data showed that PD-1H surface protein is expressed in normal myeloid cells but rarely in resting T cells in AML BM (FIG. 1C). More importantly, PD-1H was highly expressed on CD34+ and CD33+ AML blasts in BM from AML patients, consistent with the IHC findings (FIGS. 1A & 1C, Tables 1 & 2). In contrast, normal CD34+ progenitor cells in BM from healthy donors exhibited minimal expression of PD-1H cell surface protein (FIG. 1C). We quantified the expression level of PD-1H cell surface protein on AML blasts from 25 AML patients to compare with that of CD34+ progenitor cells from healthy donors (Table 2). The mean fluorescence intensity (MFI) of PD-1H in AML blasts (N=25) was significantly higher than the MFI of PD-1H in normal CD34+ progenitors from all healthy donors (N=6) (FIG. 1D). Consistent with database analyses of PD-1H mRNA transcript, M4 and M5 AML were the subtypes with higher expression of PD-1H surface protein (FIGS. 1E & 1G and Table 2), and t(8;21) AML blasts had very low expression of PD-1H (FIGS. 1E & 1F and Table 2). We also found that PD-1H expression was higher in monocytic leukemia cell lines (THP1, U937, MOLM14) than in leukemia cell lines containing RUNX1-RUNX1T1 (Kasumi1) and PMLRARa (HL-60, NB40).

Collectively, these data suggest that PD-1H surface protein is highly expressed on AML blasts, but not on normal CD34+ progenitor cells; that PD-1H surface expression is higher in monocytic leukemia than in non-monocytic leukemia and in monosomy or complex karyotype AML than in t(8;21) AML; and that high expression of PD-1H in AML BM results mainly from expression of PD-1H by AML blasts in addition to PD-1H expression on normal myeloid cells.

### AML Surface PD-1H Induces Immune Evasion

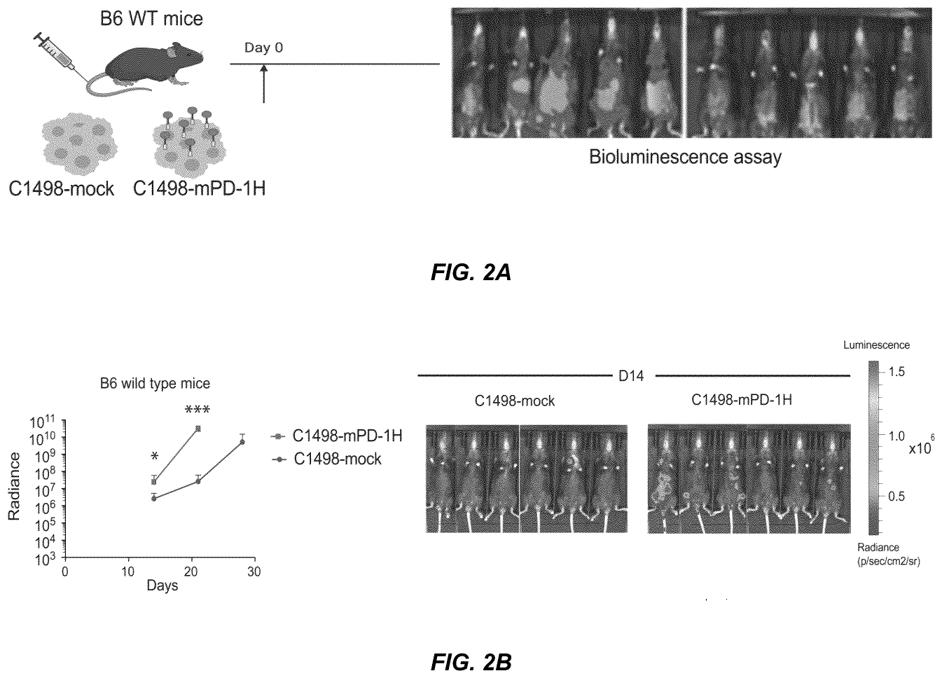

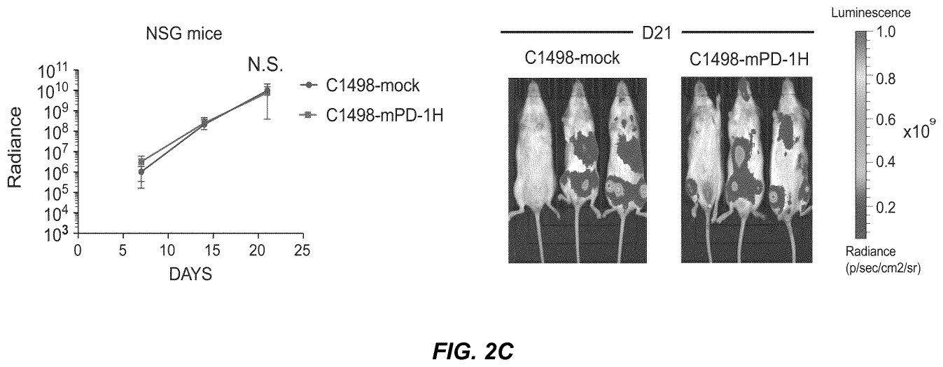

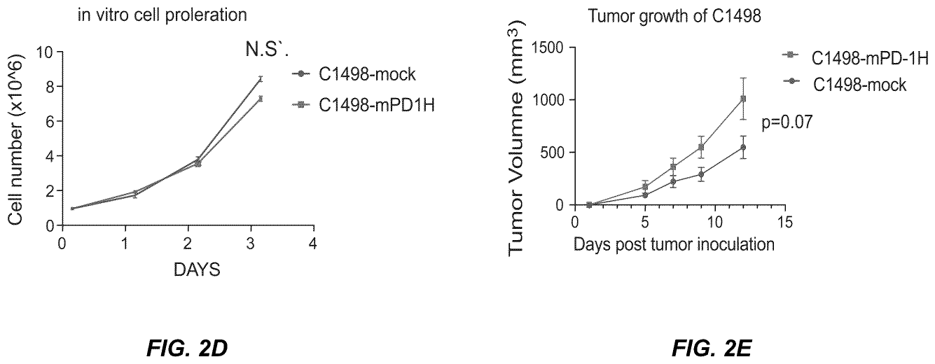

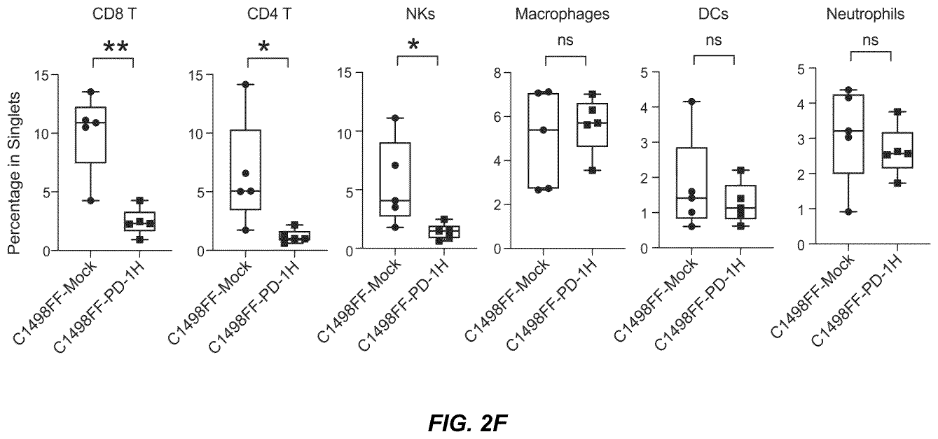

Since PD-1H expressed on myeloid cells can work as a co-inhibitory ligand to negatively modulate T cell activation and function, we hypothesized that PD-1H on the AML cell surface may induce immune evasion. We assessed AML progression in vivo in a syngeneic AML transplant murine model. C1498 is a murine myeloid leukemia cell line that developed spontaneously in a C57BL/6 (B6 hereafter) mouse (Dunham L J, J Natl Cancer Inst. 1953 13(5): 1299-1377). PD-1H expression in C1498 parental cells is undetectable. We intravenously (i.v.) injected C1498FF cells (engineered to express luciferase) transduced with a PD-1H expression lentiviral plasmid (C1498FF-mouse PD-1H (mPD-1H)) or C1498FF cells transduced with a control lentiviral plasmid (C1498FF-mock) in syngeneic B6 mice to assess tumor growth in vivo using a bioluminescence assay (FIG. 2A). Interestingly, in vivo tumor growth of C1498FF-mPD-1H was significantly faster than that of C1498FF-mock cells in wild-type (WT) B6 mice (Mean radiance of C1498FF-mock vs. C1498FF-PD-1H on day 21: 2.6×10⁷ vs. 3.2×10¹⁰, N=7, P=0.0002) (FIG. 2B). To determine whether faster in vivo proliferation of C1498FF-mPD1H cells is associated with immune evasion, we transplanted either C1498FF-mPD-1H or C1498FF-mock cells into immunodeficient NOD-scid-IL2Rgamma^{null} (NSG) mice. C1498FFmPD-1H and C1498FF-mock tumors grew equally in NSG mice, suggesting that AML blast PD-1H may promote disease progression by immune evasion (FIG. 2C). In addition, these two cell lines grew with similar speed in culture (FIG. 2D). Interestingly, we also transplanted C1498FF-mPD-1H or C1498FF-mock cells in PD-1H knockout (KO) B6 mice and found that, similar to the observation in WT B6 mice, the C1498FF-PD-1H tumor growth was still faster than that of C1498FF-mock cells (Mean radiance of C1498FF-mock vs. C1498FF-PD-1H on day 21: 1.4×10⁵ vs. 4.3×10⁷, N=7, P=0.01). These findings suggested that the acceleration of PD-1H+ AML in immunocompetent mice is not dependent on PD-1H expression on the host cells.

B7-1 (CD80), a well-known co-stimulatory ligand, provides a strong anti-tumor effect via engagement with CD28 on anti-tumor T cells (Boyer M W, et al. Blood. 1997 89(9):3477-3485; Chen L, et al. Cell. 1992 71(7): 1093-1102). We transplanted either C1498FF-B7-1 or B7-1/mPD-1H co-expressing (C1498FF-B7-1-mPD-1H) cells into WT B6 mice to assess in vivo tumor growth. Interestingly, C1498FF-B7-1-mPD-1H tumor grew faster in vivo than C1498FFB7-1 tumor (Mean radiance of C1498FF-B7-1 vs. C1498FF-B7-1-PD-1H on day 21: 4.6×10⁵ vs. 5.3×10⁷, N=3 per group, P=0.01). These data suggest that the immune evasion effect of AML blast PD-1H can override the immune activation effect of B7-1.

To facilitate the study of the immune components in a PD-1H-positive versus a PD-1H-negative AML microenvironment, we established a subcutaneous (s.c.) AML tumor model. Either C1498FF-mPD-1H or C1498FF-mock cells were inoculated s.c. in B6 mice. Consistent with the result when i.v. injected, C1498FF-mPD-1H s.c. tumors also grew faster than C1498FF-mock tumors (FIG. 2E) even though the difference was not statistically significant (Mean size of C1498FF-mock tumors vs. C1498FF-PD-1H tumors on day 12: 547 vs. 1,011 mm³, P=0.07). The tumors were removed on day 12 after inoculation and infiltrating immune cells were profiled by a mass cytometry (CyTOF), a single cell analysis tool. C1498FF-mPD-1H tumors had significantly lower immune cell infiltration, especially CD4⁺ T cells, CD8⁺ T cells, natural killer (NK) cells. Of note, the infiltration of macrophages and neutrophils in C1498FF-m-PD-1H tumors was not significantly different than that in C1498FF-mock tumors (FIG. 2F), indicating a selective inhibition by PD-1H on lymphoid cells. To determine whether PD-1H on AML cells suppresses T cells and PD-1H blockade reverses AML PD-1H mediated T cell inhibition, we transplanted C1498FF-mPD-1H in PD-1H KO mice and treated them with either PD-1H blocking antibody (13F3) or isotype control. While 13F3 suppressed AML proliferation in vivo, T cell quantity in AML BM and spleen increased in mice treated with 13F3 compared with those treated with isotype control.

In addition to overexpressing PD-1H in C1498 cells, we also performed PD-1H knockdown in murine myeloid leukemia cell line WEHI3, which constitutively expresses PD-1H, using short hairpin RNA (shRNA) (WEHI3-PD-1H^{low} vs. WEHI-PD-1H^{high}) and tested the effect of PD-1H knockdown on leukemia growth in vivo. Consistent with the result of the C1498-mPD-1H s.c. tumors, WEHI3-mPD-1H_{high} tumors grew significantly faster than WEHI3-mPD-1H^{low} tumors (p<0.05). Meanwhile, IHC studies suggested that WEHI3-mPD-1H^{high} tumors have lower infiltration of T cells than WEHI3-mPD-1H^{low} tumors.

Altogether, these data suggest that AML blast PD-1H induces immune evasion by suppressing infiltrating T cells in the leukemia microenvironment and thereby promotes leukemia growth.

### Host-Derived PD-1H Also Mediates Immune Evasion in AML

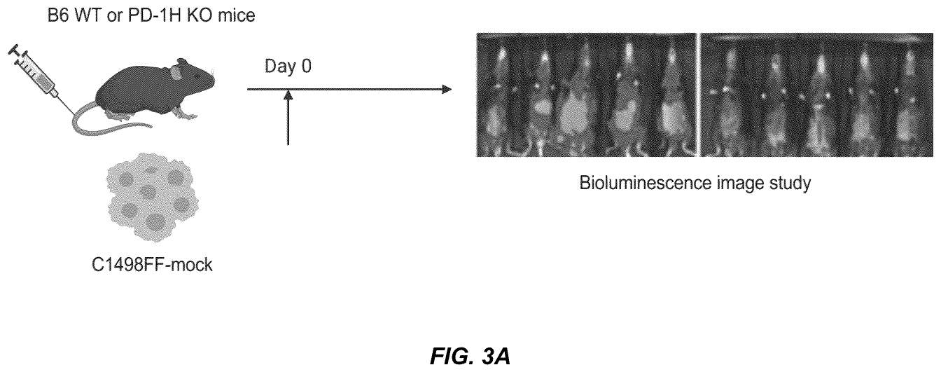

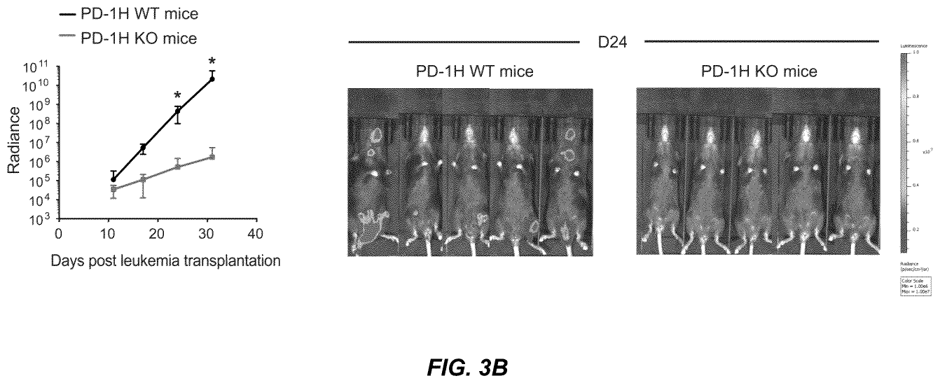

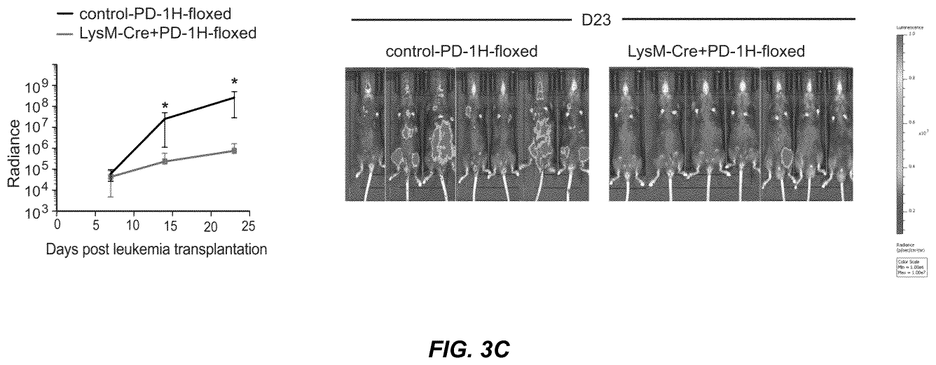

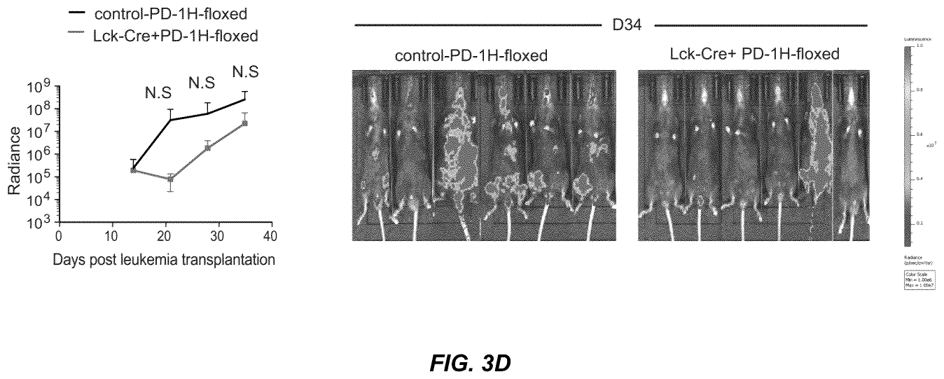

While PD-1H is expressed on AML blasts and acts as a ligand to suppress T cell activation as demonstrated above, PD-1H is also expressed on host immune cells, including T cells and macrophages (Flies D B, et al. J Immunol. 2011 187(4): 1537-1541; Lines J L, et al. Cancer Res. 2014 74(7): 1924-1932). We hypothesized that PD-1H on host immune cells (immune cell surface PD-1H) may also contribute to immune evasion in AML. To test this, C1498FF-mock cells were i.v. transplanted into PD-1H KO or WT B6 mice, and tumor growth was monitored using bioluminescence in vivo (FIG. 3A). The genetic depletion of PD-1H in KO mice conferred significant anti-leukemic effects (Mean radiance in PD-1H WT vs. PD-1H KO mice on day 24: 4.4×10⁸ vs. 5.1×10⁵, N=5, P=0.04) (FIG. 3B). This led to improved survival, compared with PD-1H WT mice (median survival of PD-1H WT mice vs. PD-1H KO mice: 33 vs. 65 days, p=0.006). BM and spleen from PD-1H KO or WT AML mice were assessed for the quantity of immune cell subsets. The quantity of macrophages and granulocytes were significantly increased in PD-1H KO AML mice compared with WT AML mice. In addition, the ratio of pro-inflammatory macrophages to anti-inflammatory macrophages was higher in PD-1H KO mice than WT mice. The quantity of macrophages and granulocytes or the ratio of pro-inflammatory macrophages to anti-inflammatory macrophages were not significantly different between naive PD-1H KO or WT mice. Other cell subsets including regulatory T cells (CD4⁺CD25^{hi}FoxP3⁺), and CD4 and CD8 T cells were not changed while NK cells increased, and dendritic cells decreased in PD-1H spleen. Anti-leukemia effect of host immune PD-1H deletion was recapitulated in PD-1H WT mice treated with anti-mPD-1H mAb (Clone 13F3) although not statistically significant when compared with isotype control treated mice (Mean radiance of anti-mPD-1H Ab (13F3) vs. isotype on day 14: 1.4×10⁶ vs. 4.4×10⁷, N=5, P=0.07).

To further dissect the role of host-derived PD-1H, we generated lineage-specific KO mice that do not express PD-1H in T cells (Lck-Cre⁺PD-1H^{fl/fl} vs. Lck-Cre-PD-1H^{fl/fl}) or in myeloid cells (macrophages, granulocytes) (LysM-Cre⁺PD-1H^{fl/fl} vs. LysM-Cre-PD-1H^{fl/fl}). Following i.v. transplantation with C1498FF cells, we assessed tumor growth in these KO mice and littermate controls using bioluminescence in vivo. We found that the tumor growth was significantly inhibited by myeloid cell-specific genetic deletion of PD-1H, compared with littermate controls (Mean radiance in Cre-LysM-PD-1H_{fl/fl} vs. Cre-LysM₊PD-1H_{fl/fl} on day 23: 1.7×10⁹ vs. 1.7×10⁶, N=9, P=0.03) (FIG. 3C). T cell-specific genetic deletion of PD-1H showed a trend toward potent anti-leukemia effects, but it was not statistically significant (Mean radiance in Cre-Lck-PD-1H^{fl/fl} v.s Cre-Lck+PD-1H^{fl/fl} on day 34: 2.5×10⁸ vs. 2.2×10⁷, N=6, P=0.1). (FIG. 3D). Taken together, our results support critical role of both AML blast and host myeloid cell-derived PD-1H on immune evasion to promote AML growth.

### Anti-PD-1H mAb Reverses Immune Evasion in AML

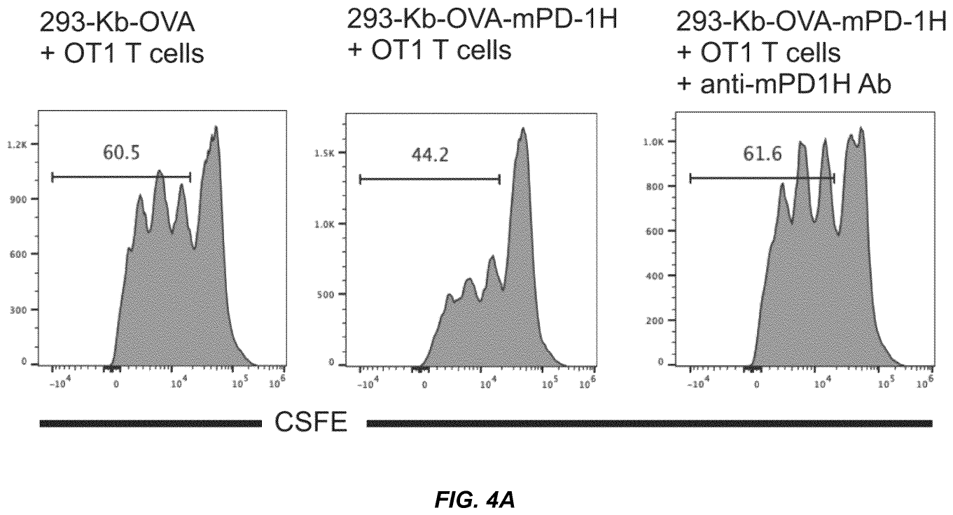

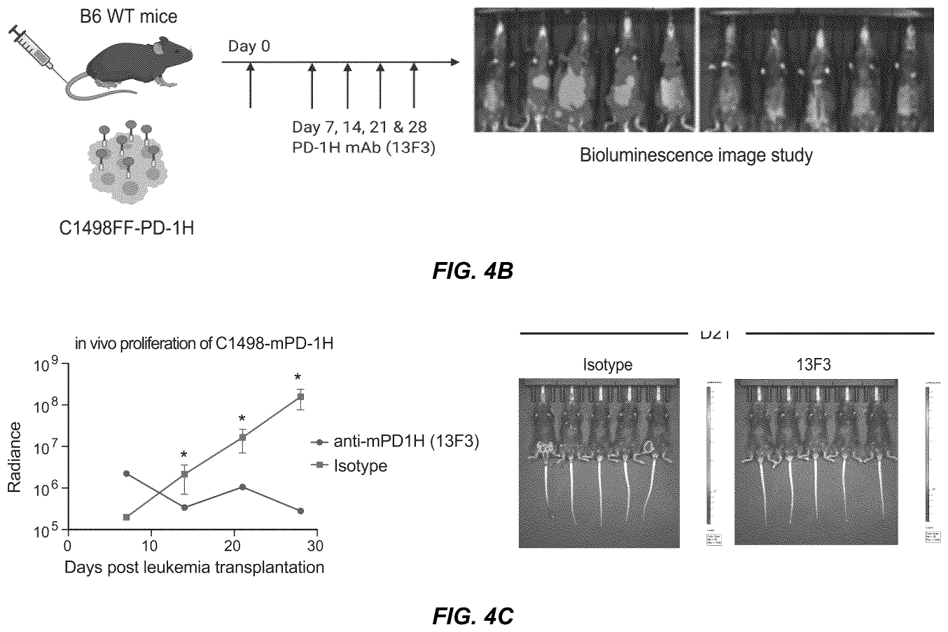

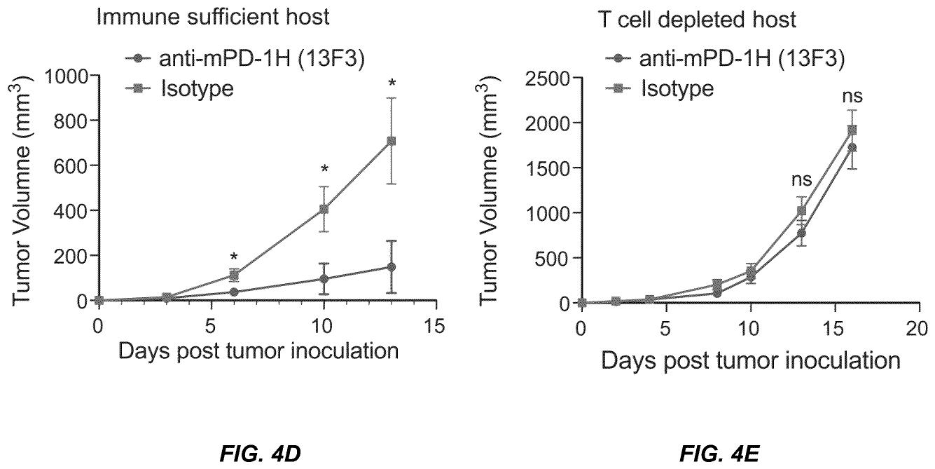

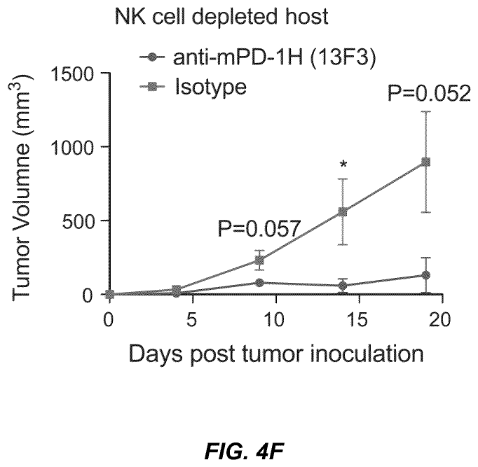

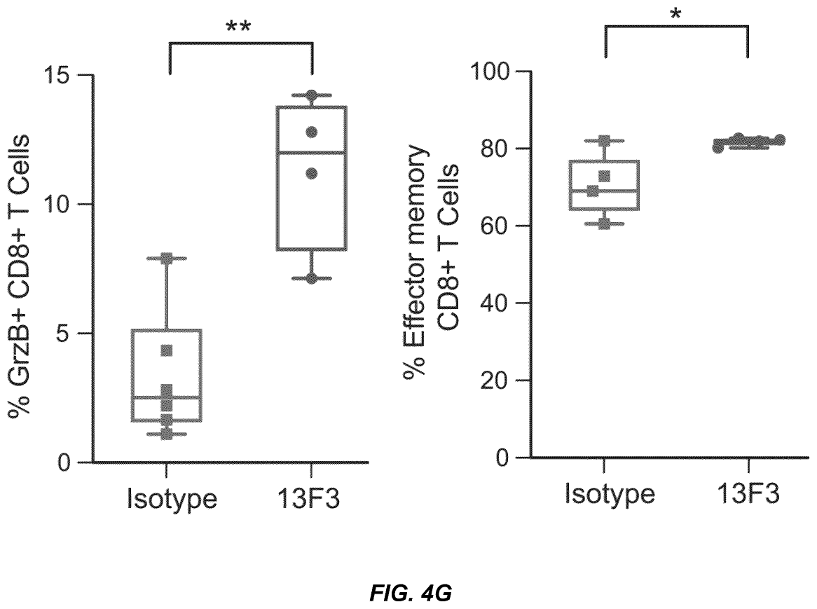

In the context of an immune suppressive role of AML blast- and host cell-derived PD-1H, a maximal therapeutic effect may be achieved with a specific mAb to block PD-1H systemically. 13F3 is a mPD-1H-specific mAb which was shown to effectively block the PD-1H pathway and enhance immune responses in mouse tumor and autoimmune disease models (Wang L, et al. J Exp Med. 2011 208(3):577-592; Le Mercier I, et al. Cancer Res. 2014 74(7): 1933-1944; Sergent P A, et al. Lupus. 2018 27(2):210-216). We first validated the blocking effect of anti-mPD-1H in an in vitro APC/T cell activation assay. In this assay, a HEK293T-K^{b}-ovalbumin (OVA) cell line (293T-K^{b}OVA) stably expressing the mouse H-2K^{b} molecule and the chicken OVA 257-264 peptide (OVA²⁵⁷⁻²⁶⁴) is used as the APC to activate mouse CD8₊ OT-1 TCR transgenic T cells (Wang J, et al. Cell. 2019 176(1-2):334-347.e12). Compared to 293T-K^{b}OVA cells, 293T-KbOVA cells stably expressing murine PD-1H on their cell surface (293T-K^{b}OVA-PD-1H) induced much less OT-1 T cell proliferation. However, in the presence of 13F3 mAb, 293TK^{b}OVA-PD-1H cells' inhibitory effect was completely blocked (FIG. 4A).

The effect of the PD-1H mAb on AML growth in vivo was first tested using the C1498FFmPD-1H AML model. After C1498FF-mPD-1H AML cells were transplanted into B6 WT mice either i.v. or s.c., the 13F3 mAb or a control mAb was given to mice. 13F3 treatment dramatically slowed down the in vivo growth of both disseminated C1498FF-mPD-1H AML cells and s.c. tumors (Mean radiance of 13F3 vs. isotype control on day 28: 2.8×10⁵ vs. 3.3×10⁷, N=5 per group, P=0.02; Mean size of C1498FF-PD-1H tumors in mice treated with 13F3 vs. with isotype control on day 13: 708.8 vs. 148.6 mm³, N=6, P<0.05) (FIGS. 4B-4D). Depletion of T cells by CD4 and CD8 mAbs completely abolished the anti-leukemic effect of 13F3 in WT B6 mice, whereas NK cell depletion had no effect (FIGS. 4E & 4F). These findings suggest that 13F3 treatment inhibits C1498FF-mPD-1H leukemia growth by enhancing T cell immunity but not NK cells or antibody-dependent cell-mediated cytotoxicity (ADCC) which is largely mediated by NK cells. Further analysis of T cell subsets in tumor tissues by mass cytometry revealed that the percentages of Granzyme B₊CD8₊ T cells as well as effector memory phenotype (CD44⁺CD62L⁻) CD8₊ T cells were significantly increased, albeit there was no significant increase of total CD8⁺ or CD4⁺ T cell infiltration in PD-1H-positive leukemia after 13F3 treatment compared with controls (FIG. 4G). These data indicate that PD-1H blockade improves the quality of the T cell response rather than augmenting T cell infiltration in this leukemia model.

In the studies described above, we found both AML surface PD-1H and host-derived PD-1H can induce immune evasion in AML. It was unclear whether the therapeutic effect of PD-1H mAb is mediated by either the blocking of PD-1H on AML blasts, or PD-1H on the host cells or both. To test the effect of anti-mPD-1H mAb (13F3) in the absence of host cell-derived PD-1H, C1498FF-PD-1H AML cells were s.c. or i.v transplanted into B6 PD-1H KO mice, and mice were treated with 13F3 or control mAb. We found that 13F3 significantly reduced C1498FFmPD-1H AML growth in PD-1H KO mice, with an effect similar to that seen in B6 WT mice (FIG. 4E). Similar results were also observed using a different anti-mPD-1H mAb (clone mam82) (Flies D B, et al. J Clin Invest. 2014 124(5): 1966-1975) and using another leukemia model (WEHI3) in PD-1H KO mice, where PD-1H blockade was associated with increased T cell infiltration. To exclude the possibility that the mAb may directly deliver a death signal into AML cells through cell surface PD-1H, we also assessed in vivo growth of C1498 engineered to express PD-1H without its intracellular domain (C1498FF-PD-1H-A). Anti-mPD-1H mAb could also reduce C1498FF-mPD-1H-A growth in vivo, suggesting that mAb was not affecting signaling within AML cells, but rather blocking the effect of AML blast PD-1H on T cell immune evasion.

As described earlier, 13F3 treatment had a modest effect on in vivo growth of disseminated C1498FF-mock AML tumors in WT B6 mice. These data further confirmed our finding that host-derived PD-1H also contributes to immune evasion in AML. Because C1498 cells do not express PD-1H, the anti-tumor effect of 13F3 could be attributed to the blockade of host-derived PD-1H.

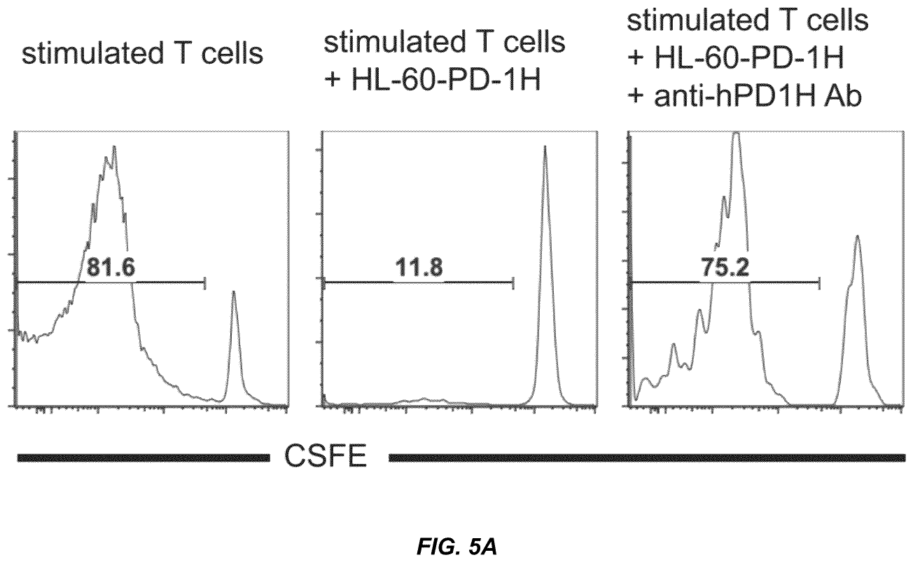

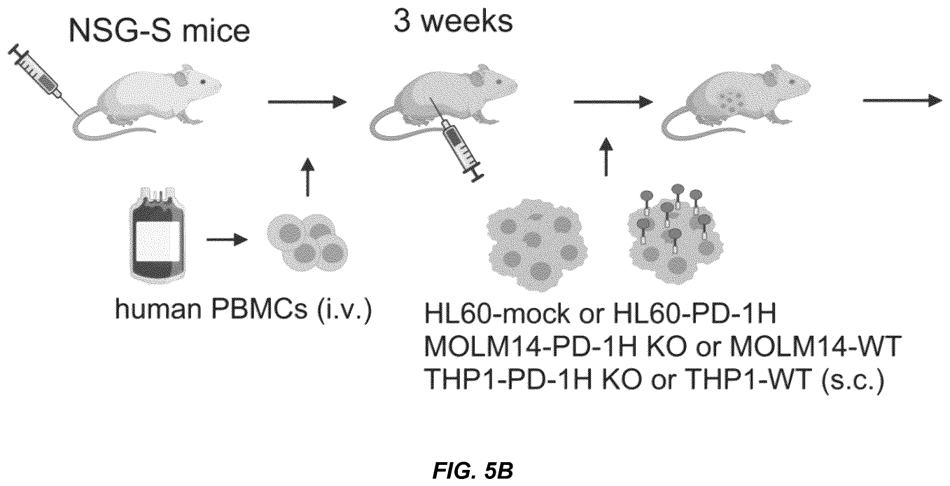

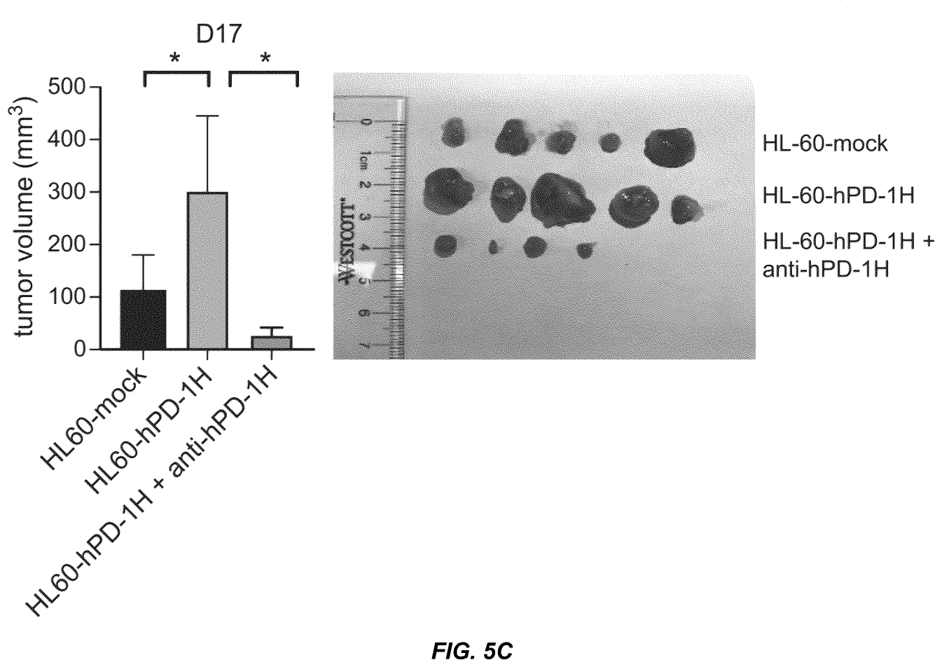

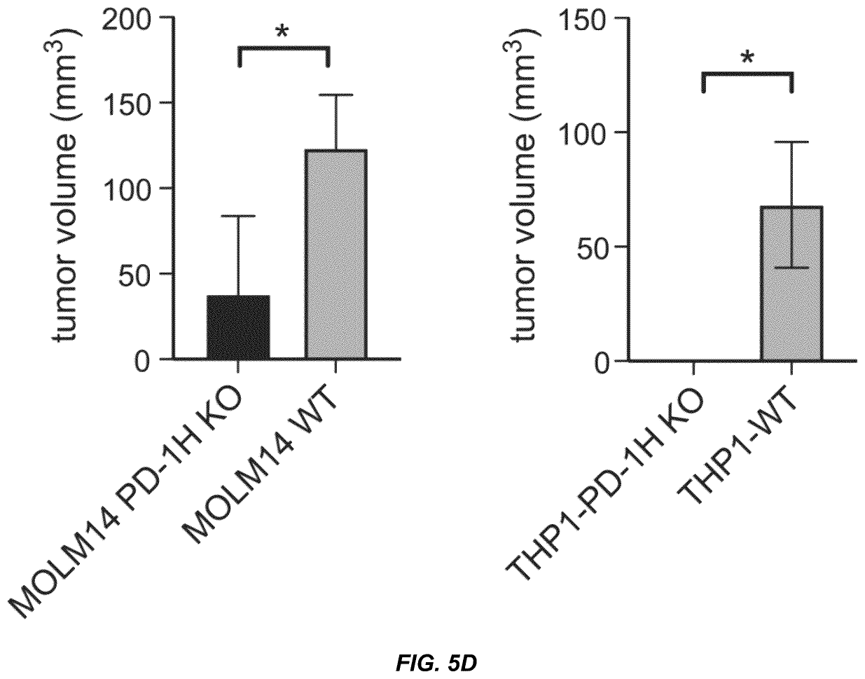

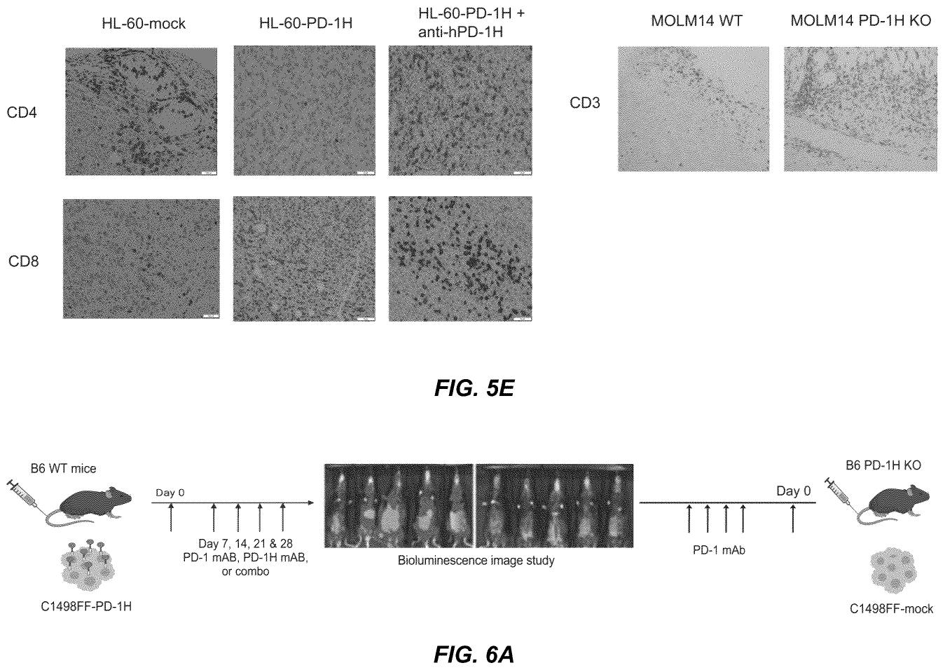

In addition to the murine AML model, we tested whether human AML blast PD-1H could also induce immune evasion using a humanized AML model. In addition to this mouse T cell activation assay (FIG. 4A), we also performed an in vitro human T cell activation/proliferation assay by stimulating human T cells with anti-CD3 mAb in the presence of HL-60-hPD-1H or HL-60-mock cells. We found that human T cell proliferation was significantly inhibited by PD-1H on HL-60 cells (FIG. 5A). Likewise, in the presence of a mAb against hPD-1H (Clone MIH65), T cell suppression by HL-60-hPD-1H was reversed (FIG. 5A). To test whether anti-hPD-1H mAb reverses T cell inhibition induced by PD-1H on human primary AML blasts, we attempted in vitro T cell activation/proliferation assay in human primary AML BM cells containing PD-1H expressing blasts. T cell proliferation by polyclonal stimulation with anti-CD3/CD28 was marginal in primary AML BM cells. However, the addition with anti-hPD-1H mAb induced more significant T cell proliferation (especially CD4 T cells) than isotype control.

Using similar strategies as shown in the murine cell lines, we overexpressed PD-1H in the PD-1H negative human leukemia cell line HL-60 or knocked out PD-1H in the PD-1H positive human leukemia cell lines MOLM14 and THP1. HL-60-hPD-1H or HL-60-mock cells were s.c. injected into immunodeficient NSG-SGM3 (NSG-S) or NSG mice reconstituted with allogeneic human T cells (FIG. 5B). Two weeks after leukemia cell inoculation, we sacrificed mice and assessed the size of leukemic tumors. The size of HL-60-hPD-1H tumors (PD-1H+) was significantly bigger than that of HL-60-mock tumors (PD-1H−) (FIG. 5C). Consistent with these findings, other PD-1H+ AML tumors (MOLM14-WT, THP1-WT) also grew bigger than PD-1H− AML tumors (MOLM14-PD-1H KO, THP1-PD-1H KO) (FIG. 5D). At the same time, IHC studies showed fewer infiltrating T cells within HL-60-hPD-1H tumors and MOLM 14-WT tumors than HL-60-mock and MOLM14-PD-1H KO tumors, respectively (FIG. 5E). But we could not assess T cell infiltration in THP1-PD-1H KO tumors because all tumors were rejected. We determined the effect of an anti-hPD-1H mAb in a humanized AML model (FIG. 5B). The treatment with anti-hPD-1H mAb (clone MIH65) significantly reduced the size of HL-60-hPD-1H tumors (FIG. 5C), accompanied by increased T cell infiltration (FIG. 5E). Therefore, our findings further extend and validate the results in syngeneic mouse leukemia models showing that PD-1H mAb can reverse the immune evasion induced by PD-1H.

PD-1H Blockade Confers a Synergistic Anti-Leukemic Effect with PD-1 Blockade

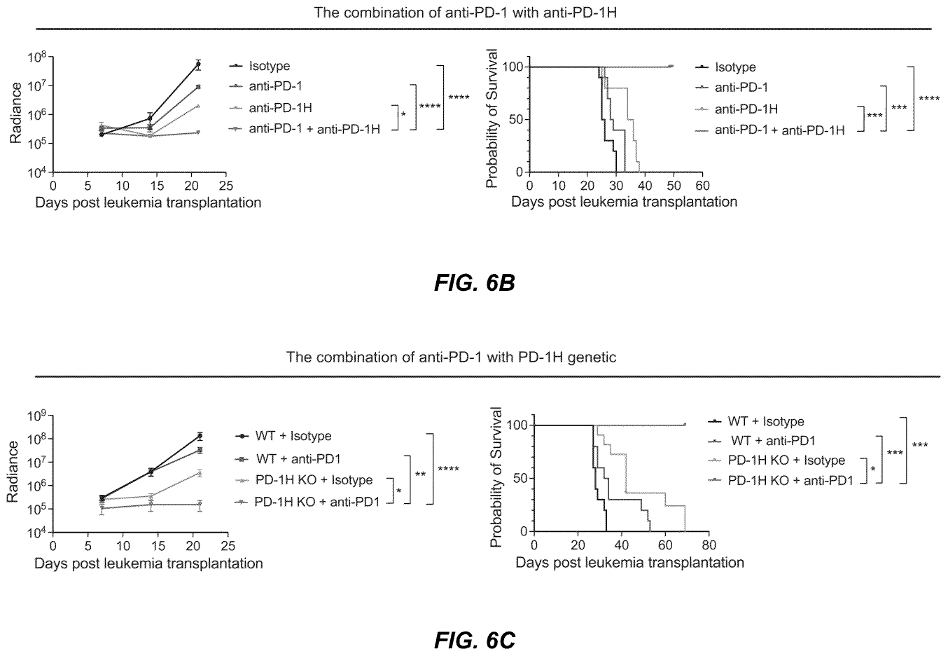

Consistent with the prior preclinical studies in which PD-1 or PD-L1 blockade had antileukemia effect (70, 71), we also observed modest reduction of in vivo growth of C1498FFmPD-1H leukemia in WT mice following anti-mouse PD-1 (mPD-1) mAb treatment compared with an isotype control (FIG. 6A). Interestingly, when C1498FF-mPD-1H-bearing mice were treated with anti-mPD-1 mAb along with anti-mPD-1H mAb, a synergistic anti-leukemia effect was observed, compared with either anti-mPD-1 mAb or anti-mPD-1H mAb monotherapy (Mean radiance of isotype 5×10⁷, anti-PD-1 9×10⁶, anti-PD-1H 2×10⁶, combination of anti-PD1 with anti-PD-1H 2.3×10⁵ on day 21, N=10 per group, *<0.05, ***<0.001, ****<0.001) (FIG. 6B). This synergistic anti-leukemia effect led to longer survival (mean survival for isotype in WT mice, 25.5 days, for anti-PD-1 in WT mice, 28.5 days, for anti-PD-1H, 35 days, for anti-PD-1+anti-PD-1H, undefined. All p-value compared with anti-PD-1+anti-PD-1H*<0.05, ***<0.001, ****<0.001) (FIG. 6B). To confirm these data, we transplanted C1498FF-mock cells into PD-1H KO or WT mice (FIG. 6A). In this model, PD-1H is absent in host immune cells as well as on AML cells, analogous to treatment with effective PD-1H blockade. Following anti-mPD-1 mAb treatment, in vivo AML growth was assessed using bioluminescence. Consistent with the combination treatment with anti-mPD-1H and anti-mPD-1 mAbs, anti-mPD-1 mAb treatment conferred a synergistic anti-leukemia effect in PD-1H KO mice compared with anti-mPD-1 mAb treatment in WT mice or isotype treatment in PD-1H KO mice, and led to a longer survival (mean survival for isotype in WT mice, 32 days, for anti-PD-1 in WT mice, 49 days, for isotype in PD-1H KO, 60 days, for anti-PD-1 in PD-1H KO mice, undefined. All p-value compared with anti-PD-1 in PD-1H KO mice <0.05) (FIG. 6C). Our results showed a synergistic effect of blocking both PD-1H and PD-1 pathways in this model.

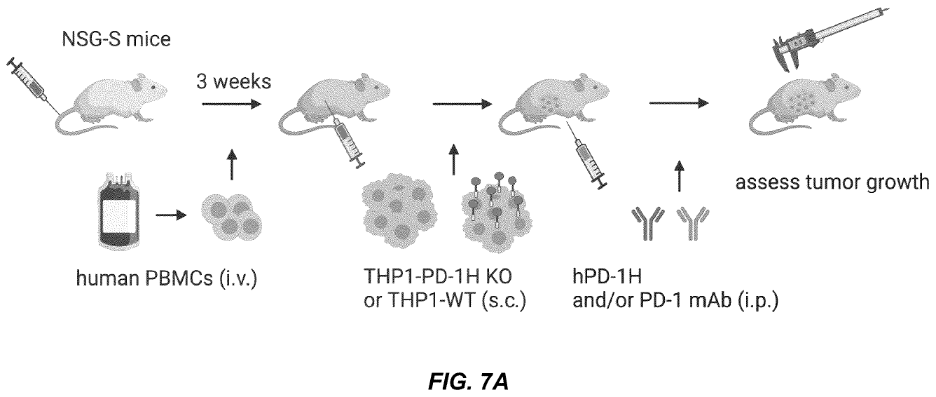

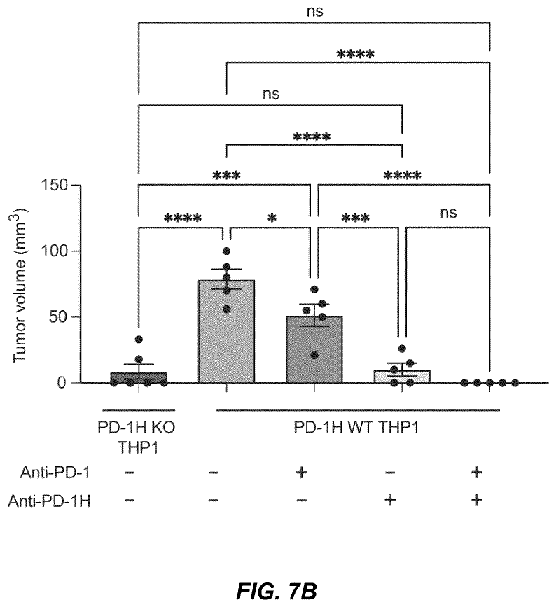

To test a synergistic anti-leukemia effect of anti-hPD-1H mAb with anti-human PD-1 (hPD-1) mAb, we used a humanized AML model again (FIG. 7). THP1-WT (PD-1H+) cells were s.c. injected into immunodeficient NSG mice reconstituted with allogeneic human T cells (FIG. 7). Following anti-hPD-1H and/or anti-hPD-1 mAb, we assessed the size of leukemic tumors. Consistent with the observation in FIG. 4J, anti-hPD-1H mAb significantly decreased the size of tumors but anti-hPD-1 mAb did not suppress the AML tumor growth (FIG. 7). Interestingly, the combination of anti-hPD-1H mAb and anti-hPD-1 mAb completely rejected AML tumors (Mean AML tumor volume±SEM (on day 9) was 44.4±23.3 in THP WT treated with Isotype, 33.6±16.8 in THP1 WT treated with anti-PD-1, 14.5±9.8 in THP1 WT treated with anti-PD-1H, 11.1±11.1 in THP1 WT treated with the combination of anti-PD-1 with anti-PD-1H, (n=5). *<0.05, ***<0.001, ****<0.001) (FIG. 7). These data suggest that anti-hPD-1H mAb treatment confers a synergistic anti-leukemia effect with anti-hPD-1 mAb in human AML.

### DISCUSSION

In this report, we provide evidence that AML blast PD-1H is inhibitory for intrinsic T cell-mediated immune responses against AML and therefore may contribute to escape of AML from immune destruction. We also demonstrated that PD-1H on immune myeloid cells in AML BM may contribute to immune evasions. In this context, blockade of PD-1H by a specific mAb to eliminate its function could improve anti-AML immunity and induce the regression of AML. Finally, we showed that, while the effect of PD-1 blockade is modest in a syngeneic AML mouse model and a humanized AML mouse model, combination PD-1/PD-1H blockade confers a synergistic anti-leukemia effect, leading to the regression of established AML. Our findings provide experimental evidence showing the role of PD-1H in inhibiting anti-AML immunity and implicating a potential new target for AML immunotherapy.

Anti-PD-1 therapy showed unprecedented therapeutic effects on subsets of many different cancers, mainly in solid tumors (Vaddepally R K, et al. Cancers (Basel). 2020 12(3): E738). Early data from clinical trials did show marginal clinical response in myeloid malignancies, such as AML or myelodysplastic syndrome (MDS), when using mAbs targeting CTLA4 or PD-1/PD-L1 as single agents (Daver N, et al. Cancer Discov. 2019 9(3):370-383; Garcia-Manero G, et al. Blood. 2016 128(22):344-344; Davids M S, et al. N Engl J Med. 2016 375(2): 143-153; Zeidan A M, et al. Clin Cancer Res. 2018 24(15):3519-3527). Since hematologic malignancies do not have obvious tumor immune microenvironment (TIME) as solid tumors (Kim T K, et al. Trends Immunol. 2018 39(8):624-631; Zhang Y, et al. JAMA Oncol. 2016 2(11): 1403-1404; Kim T K, et al. Nat Rev Drug Discov. 2022 21(7):529-540), the underlying immune evasion mechanisms for the poor response to immune checkpoint blockades in AML/MDS could be different. Recently, Williams et al. showed that T cells are present and phenotypically changed in the AML BM, and that the phenotype bears similarity to the exhausted or persistently activated phenotype (PD-1+, OX40+, TIM3+, LAG3+) seen in other cancers (Williams P, et al. Cancer. 2019 125(9): 1470-1481). A study by Lamble et al. suggest that anti-PD-1 mAb converted “exhausted” T cells back to active effector cells in AML ex vivo (Lamble A J, et al. Proc Natl Acad Sci USA. 2020 117(25): 14331-14341). Therefore, the marginal clinical response to anti-PD therapy in AML might be associated with ‘in vivo’ tumor evasion mechanisms other than the PD-1/PD-L1 pathway. Another possibility is that the exhausted T cells are not responsible for immune evasion in AML. Several studies reveal that dysfunctional T cells in cancer may display different phenotypes than exhaustion and these phenotypes include but are not limited to anergy, ignorance, and burn-out (Sanmamed M F, et al. Cancer Discov. 2021 candisc.0962.2020). PD-1H has been shown to function as both receptor and ligand. As a ligand, it can deliver potent suppressive signals to T cells by shutting down both proximal and downstream T cell receptor signals. We and others showed that PD-1H, upon binding to T cells, decreased phosphorylation of LAT, SLP76, PLCγ-1, Akt, and Erk 1/2 (Han X, et al. Science Translational Medicine. 2019 11(522); Liu J, et al. Proc Natl Acad Sci USA. 2015 112(21):6682-6687). Blando J et al, showed that PD-1H was superior in suppressing T cell cytokine release (IFN-γ, TNFα) to PD-L1 when cocultured with pancreatic tumor-infiltrating lymphocytes (Blando J, et al. Proc Natl Acad Sci USA. 2019 116(5): 1692-1697). These results indicate that the PD-1H signaling axis is a powerful immunomodulatory pathway. PD-1H may execute its inhibitory function via its receptor(s) on T cells, which remains to be fully elucidated. Our recent analysis of PD-1H molecular structure reveals a unique non-canonical immunoglobulin V-like region which may allow multiple binding partner interactions (Slater B T, et al. Proc Natl Acad Sci USA. 2020 117(3): 1648-1657). PD-1H appears to bind PD-1H, VSIG3 and more recently, P-selectin glycoprotein ligand-1 (PSGL-1) (Johnston R J, et al. Nature. 2019 574(7779):565-570; Wang J, et al. Immunology. 2019 156(1):74-85). Interestingly, the binding of PD-1H to PSGL-1 is dependent on acidic pH (Johnston R J, et al. Nature. 2019 574(7779):565-570), which is more common in solid tumors, and its role in our system is unknown. This question will be tested in future studies to assess pH, PSGL-1 expression, colocalization of PSGL-1 with PD-1H in human AML BM, and functional assessment of PSGL-1 in AML.

We demonstrated that AML BM has high expression of PD-1H and that PD-1H expression is higher in monocytic and myelomonocytic AML cells than non-monocytic AML cells and healthy donor BM. Also, PD-1H expression is higher in poor risk complex karyotype AML than in t(8;21) and t(15;17) good risk AML. The differential expression of PD-1H mRNA observed in TCGA AML correlates with the expression of AML surface PD-1H assessed by flow cytometry. For example, PD-1H expression on monocytic blasts is higher than on nonmonocytic blasts and PD-1H expression on complex karyotype AML blasts is higher than on t(8;21) good risk AML blasts (no data acquired for t(15;17) AML). This suggests that PD-1H targeting can be more effective in monocytic leukemia. It remains to be elucidated what regulates the expression level of PD-1H in different types of leukemia blasts; possibilities include altered signaling, epigenetic modulation, or cytokine-related modulation. We also found PD-1H expression in AML is correlated with poor survival. Worse survival in PD-1H^{high} AML may result from immune evasion induced by PD-1H, but other confounding factors that impact survival, including cytogenetics and certain genetic mutations, cannot be completely excluded to explain the worse survival in PD-1H^{high} AML.

We demonstrated the role of AML blast PD-1H on immune evasion in vitro and in vivo in a syngeneic AML model as well as in a humanized mouse model. Our results suggest that AML blast PD-1H acts as a ligand that suppresses T cell activation. It remains to be elucidated if AML blast PD-1H also suppresses the activation of innate immune cells such as macrophages, granulocytes, and NK cells. To our knowledge, this is one of few studies demonstrating that a co-inhibitory ligand on AML blasts induces immune evasion and that its blockade reverses the immune evasion in AML. In addition, we also showed the role of immune cell PD-1H in immune evasion in AML in mice with the full or conditional genetic deletion of PD-1H transplanted with syngeneic AML cells. Interestingly, macrophage/neutrophil PD-1H contributed more significantly to immune evasion in AML compared with T cell PD-1H. Our data is one of few studies demonstrating the significance of checkpoint molecules expressed on immune myeloid cells in cancer immune evasion, beyond CD47-SIRP1 alpha and PD-1-PD-L1 (80-82). But our data cannot completely rule out the possibility that this immune evasion in AML is from PD-1H on myeloid derived suppressor cells, which was recently shown using in vitro experiments(54). In addition, it remains to be investigated whether macrophage/granulocyte PD-1H acts as a ligand to suppress T cell activation or acts as a receptor. Interestingly, the genetic deletion of PD-1H from macrophages/granulocytes alone without T cells did not achieve an optimal anti-leukemia effect. This suggests that macrophage/granulocyte PD-1H has a baseline immune tolerance, but breaking tolerance in innate immunity by PD-1H blockade is not enough to generate a robust anti-leukemia effect without adaptive immunity from T cells.

Our study has a couple of potential limitations. First, we used mouse myeloid leukemia cell line C1498. Syngeneic leukemia models using C1498 cells have been widely used to test the anti-leukemia effects of chemical compounds and immunotherapies (LaBelle J L, et al. Blood. 2002 99(6): 2146-2153; Mopin A, et al. J J Vis Exp. 2016 14:(116):54270; Sauer M G, et al. Cancer Res. 2004 64(11):3914-3921; Zhou Q, et al. Blood. 2011; 117(17):4501-4510). However, the genetic makeup of this cell line may not be the same as human AML, because a very low mutation rate in most primary human AML cells was observed (The Cancer Genome Atlas Research Network, 2013). Even though the data we presented here are proof of concept, it will ideally be validated using better humanized mouse models such as immune deficient mice reconstituted with autologous CD34+ progenitors followed by transplantation of primary AML blasts from the same patients. These models, however, are difficult due to competition of reconstituted T cells with the engraftment of primary AML cells as well as reactivity of human T cells to murine xenoantigens.

Here, we demonstrated that PD-1H on AML cells induces immune evasion by suppressing T cells and that host immune cell-derived PD-1H induces immune evasion in AML. PD-1H blockade reverses immune evasion, leading to inhibition of AML progression. Our data strongly suggest that PD-1H is an important immune suppressive molecule in AML that can be targeted in human AML patients.

### Methods

### Patient Samples

BM core biopsies from patients were formalin-fixed and paraffin-embedded by the Department of Pathology at Yale University and the Department of Pathology, Microbiology and Immunology at Vanderbilt University Medical Center. Tissue sectioning and IHC staining were performed by the Histology Core Service at Yale University and by the Translational Pathology Shared Resource at Vanderbilt University Medical Center.

### Animals

PD-1H KO (Genbank gene NM_028732; GenBank protein JN6-01284) mice were purchased from the Mutant Mouse Regional Resource Center at the University of California-Davis. C57BL/6 (B6) and BALB/c PD-1H KO mice were generated as previously described (Flies D B, et al. J Immunol. 2011 187(4): 1537-1541; Flies D B, et al. J Clin Invest. 2014 124(5): 1966-1975). PD-1H WT B6 mice generated from PD-1H heterozygotes were bred and maintained in conditions identical to those of PD-1H KO mice and were used as controls. Sometimes, WT B6 mice were purchased from Charles River Laboratories (Boston, MA) to confirm the data. PD-1H^{flox/flox} mice (Yoon K W, et al. Science. 2015 349(6247): 1261669) were crossed with Lck-cre (B6.Cg-Tg(Lck-cre)548Jxm/J) or LysM-cre (B6. 129P2-Lyz2^{tm(cre)/lfo}/j) mice purchased from Jackson Laboratory (Bar Harbor, ME) to generate lineage-specific conditional KO mice (T cells or myeloid cells, respectively). NSG (NOD-scid-IL2Rgamma^{null}) and NSG-SGM3 (NOD.Cg-Prkdo^{scid}Il2rgtm1Wj/Tg(CMV-IL3,CSF2,KITLG)1Eav/MloySzJ) mice were purchased from Jackson Laboratory.

### Cells

C1498 is a murine myeloid leukemia cell line that developed spontaneously in a C57BL/6 mouse. C1498FF is a stable transfectant of C1498 that expresses firefly luciferase used to assess in vivo cell proliferation. C1498FF cells were engineered to stably express mouse PD-1H using transduction with lentivirus (pLenti) expressing full-length mouse PD-1H (C1498FF-PD-1H FL) or PD-1H with deletion of its intracellular domain (C1498FF-PD-1HΔ) or with mock lentivirus (C1498FF-mock). WEHI3 is a murine myeloid leukemia cell line that originated from a BALB/c mouse (purchased from ATCC). WEHI3 cells constitutively express PD-1H. WEHI3 cells were engineered for knockdown or KO of PD-1H expression using shRNA targeting the PD-1H transcript (the Mission Library, Sigma) or CRISPR-Cas9 technologies (gRNA with Cas9 protein), respectively. HL-60 and K562 cells are human myeloid leukemia cell lines not expressing PD-1H. HL-60 or K562 cells were engineered to stably express human PD-1H using transduction with lentivirus expressing full-length human PD-1H (HL-60-PD-1H or K562-PD-1H) or a mock lentivirus (HL-60-mock or K562-mock). MOLM14 and THP1 cells are human monocytic leukemia cell lines expressing PD-1H (a gift of Martin Carroll, University of Pennsylvania).

### Flow Cytometry for Staining Human PD-1H

All human cell preparations were more than 95% viable by trypan blue exclusion. Two million thawed or fresh BM mononuclear cells were stained using mAbs conjugated with Pacific Blue, FITC, PE-Cy7, PE, PerCP-Cy5.5, APC specific for human CD3, CD11b, CD34, CD33, CD45 (BioLegend, San Diego, CA), human PD-1H (VISTA) (Clone MIH65, BD Biosciences, San Diego, CA) respectively to perform flow cytometry. After staining, cells were washed, resuspended in phosphate-buffered saline (PBS) with 1% paraformaldehyde, and analyzed in an Attune flow cytometer (Thermo Fisher, Waltham, MA) using FlowJo software (Treestar, San Carlos, CA).

### Assessment of Tumor Microenvironments Using Mass Cytometry

B6 mice were inoculated with 3×10⁶ C1498FF-mock or C1498FF-PD-1H cells. Mice were sacrificed on day 12 and tumor tissues were removed. Tumor tissue in comparable size (roughly 0.2 gram) from each mouse was used as one sample with the following treatment. Tumor tissue was homogenized and digested with collagenase IV (200 μg/mL) and DNase (20 μg/mL) for 30 min before tissue dissociation using gentleMACS Dissociator. Single-cell suspensions with 5×10⁶ total cells were then incubated with the mAb against mouse CD16/CD32 for 10 min at room temperature to block Fc receptors and subsequently stained with the metal-labeled mAb cocktail against cell surface molecules.

### Myeloid Leukemia Model for In Vivo Imaging Analyses

Approximately 3×10⁵ of C1498FF-PD-1H-FL, C1498FF-PD-1HΔ or C1498FF-mock cells in 300 μL PBS were intravenously injected into B6 WT or PD-1H KO mice. To assess in vivo proliferation of C1498FF cells, mice were intraperitoneally injected with 300 μg luciferin substrate 5 min prior to being anesthetized using an XRT-8 gas (isoflurane) anesthesia system. Anesthesia was maintained while mice were imaged for bioluminescence in a supine position using an IVIS Lumina XR in vivo imaging system (Caliper/PerkinElmer, Waltham, MA) according to the manufacturer's protocol. Briefly, luminescence detection was set to automatic with a minimum detection level of 3,000 photons. Mice were imaged on stage D at 1.5 cm height from the stage. Units were set to radiance (photons/s). Imaging and analysis were performed using Living Image software. For analysis, binning was set to 4, and minimum and maximum radiance levels were determined for optimal view and comparison between groups at each time point. Calculation of total flux was assessed (radiance or photons/s) in each pixel and then summed or integrated over the whole body (cm²×4p) by Living Image software. For experiments to evaluate the efficacy of PD-1H blockades, 3×10⁵ of C1498FF-mock or C1498FF-PD-1H-FL cells in 300 μL PBS were intravenously injected into B6 WT or PD-1H KO mice. We assessed in vivo proliferation of AML cells following intraperitoneal injection of 200 μg of anti-PD-1H mAb (clone 13F3) or hamster IgG (BioXcell) on days 0, 7, 14, and 21 post-AML cell intravenous injection. For experiments to evaluate the combination efficacy of PD-1 and PD-1H blockades, C1498FFPD-1H-FL cells in 300 μL PBS were intravenously injected into B6 WT mice. We assessed in vivo proliferation of AML cells following intraperitoneal injection of 200 μg of anti-mPD-1H mAb (clone 13F3) and/or anti-mPD-1 mAb (clone RMP1-14) or hamster IgG (BioXcell) on days 0, 7, 14, and 21 post-AML cell intravenous injection. For another experiments for combination efficacy of PD-1 and PD-1H blockades, C1498FF-mock cells in 300 μL PBS were intravenously injected into B6 WT or PD-1H KO mice. We assessed in vivo proliferation of AML cells following intraperitoneal injection of 200 μg of anti-mPD-1 mAb (clone RMP1-14) or hamster IgG (BioXcell) on days 0, 7, 14, and 21 post-AML cell intravenous injection. We repeated these experiments at least two or three times and found data were reproducible.

### Myeloid Leukemia Subcutaneous Model

B6 WT or PD-1H KO mice were inoculated s.c. in the right flank with 3×10⁶ C1498FF-mock or C1498FF-PD-1H cells. BALB/c mice were inoculated s.c. in the right flank with 0.5×10⁶ WEHI3-PD-1H shRNA or WEHI3-control shRNA cells. Tumor size was monitored every 5 days. Tumor volume was calculated as volume (mm³)=width(mm)×length (mm)×. width (mm). To test the effect of anti-PD-1H mAb on tumor growth, 200 μg of anti-mPD-1H (clone 13F3) mAb or hamster IgG (BioXcell) was intraperitoneally injected on days 0, 4, and 8 after C1498FF-PD-1H tumor inoculation.

### In Vivo Immune Cell Depletion

To deplete T cells, 250 μg of anti-CD4 (clone GK1.5) and 250 μg of CD8α (clone 53-6.7) were injected on days −4, −2, 2, 6, and 10 around tumor inoculation. To deplete NK cells, 500 μg of anti-NK1.1 (clone PK136) were injected 2 days before tumor inoculation, followed by three doses of 250 μg on post-inoculation days 2, 6, and 10.

### Humanized Myeloid Leukemia Mouse Model

Approximately 5×10⁶ human peripheral blood mononuclear cells were transplanted into NSG-S or NSG mice. After 3 weeks of transplantation, the engraftment of human cells was confirmed by human CD45 using flow cytometry. 1×10⁶ HL-60-hPD-1H or HL-60-mock cells (or 4×10⁶ THP1-WT or THP1-PD-1H KO; MOLM14 WT or MOLM 14-PD-1H KO) were s.c. injected into the flanks of the immune reconstituted NSG-S or NSG mice. Anti-human PD-1H mAb (clone MIH65) or anti-human PD-1 mAb (pembrolizumab) or isotype control Ab was intraperitoneally injected weekly from the day of tumor injection. Tumor volume was calculated as volume (mm³)=width(mm)×length (mm)×width (mm). Tumors were removed from euthanized mice to evaluate immune cell infiltration. We repeated these experiments at least two or three times and found data were reproducible.

### In Vitro Mouse OT-I CD8+ T Cell Activation by HEK293T-Kb-OVA Cell Lines.

OT-I T cells were purified from lymph nodes and spleen of Rag1KO/OT-I mice (Taconic) with EasySep Mouse CD8+ T Cell Isolation Kit (Stemcell) and labeled with 5 μM CFSE. Next, 2×10⁵ OT-I cells were cocultured with 4×10⁴ UV-radiated 293TKbOVA or 293TKbOVA-mPD-1H cells in 96 well flat bottom plate (Corning). Anti-mouse PD-1H blocking antibody 13F3 or control hamster IgG (Bio-Xcell) were added into culture at 5 μg/ml as final concentration. Three days later, cells were harvested and stained by anti-CD8 (BD). CFSE profiles on CD8+ gate were analyzed on Attune NxT cytometer (Life Technology).

### Leukemia Cell and T Cell Co-Culture Assay

Human T cells were purified from peripheral blood mononuclear cells or whole blood using an EasySep human T cell isolation kit (Stem Cell Technologies, Vancouver, Canada). Purified T cells were labeled with carboxyfluorescein succinimidyl ester (CFSE; ThermoFisher). These T cells (4×10⁴) were mixed with irradiated HL-60-hPD-1H or HL-60-mock cells with anti-human PD-1H mAb (clone MIH65) or isotype control (500 μg/mL) in a U-bottom 96-well plate.

E:T ratio was typically 4:1. Immunocult human CD3/CD28 T cell activator (25 μL/mL) (Stem Cell Technologies) and recombinant human IL-2 (50 U/mL) were added to stimulate T cells. Cells were assessed for CFSE dilution using flow cytometry.

### Graphs and Statistics

Graphs and statistical analyses were generated with GraphPad Prism 6 (GraphPad Software). Statistical analyses of survival experiments were performed using a log rank (Mantel-Cox) test; all other analyses were performed by an unpaired t-test with Welch's correction and a linear regression. The non-parametric Kruskal-Wallis test followed by Dunn's multiple comparisons test was used for identifying differences between groups in the TCGA dataset. A p-value<0.05 was considered significant.

TABLE 1
List of AML patients for immunohistochemistry

| | AML | | | | | | |
|---|---|---|---|---|---|---|---|
| | blast | Immune | Immune | Marrow | Monocytic | | |
| | PD-1H | cells PD-1H | cells PD-L1 | blast % | differen- | | |
| Diagnosis | score | positive | positive | core | tiation | Karyotype | NGS |
| AML | 1+ | Yes, MK, | Yes, MK, | 40 | No | 46, XX, t(8; 21)(q22; q22)[17]/46, XX[3] | ASXL1 |
| t(8; 21) | | myeloids | macrophages | | | | p.Glu635Argfs* |
| | | | | | | | 15 (VAF not |
| | | | | | | | available); |
| | | | | | | | PTPN11 |
| | | | | | | | pGly60Arg |
| | | | | | | | (VAF not |
| | | | | | | | available) |
| AML | 0 | Yes, MK, | Yes, MK, | 60 | No | 45, X, −X, t(8; 21)(q22; q22)[15]/46, XX[5] | Normal |
| t(8; 21) | | rare | macrophages | | | | |
| | | myeloids | | | | | |
| AML | 1+ | Yes, MK, | Yes, MK, | 20 | No | 46, XX, t(8; 21)(q22; q22)[14]/46, XX[6] | KRAS |
| t(8; 21) | | myeloids | macrophages | | | | p.Gly12Asp |
| | | | | | | | (11%) |
| AML | 1+ | Yes, MK, | Yes, MK | 70 | Yes | 46, XY, inv(16)(p13.1q22)[17]/46, XY[3] | KRAS |
| inv(16) | | myeloids | | | | | p.Gln61His |
| | | | | | | | (VAF not |
| | | | | | | | available) |
| AML | 2+ | Yes, MK, | Yes, MK, | 60 | Yes | 46, XY, inv(16)(p13.1q22)[15]/46, | FLT3-ITD |
| inv(16) | | ?myeloids | macrophages | | | XY[5] | p.Gln580_Val58 |
| | | | | | | | 1ins4 (3%); |
| | | | | | | | NRAS |
| | | | | | | | p.Gly12Asp |
| | | | | | | | (5%) |
| AML | 2+ | Yes, MK, | Yes, MK, | 60 | Yes | 46, XY, inv(16)(p13.1q22)[19]/46, | NRAS |
| inv(16) | | myeloids | macrophages | | | XY[1] | p.Gln61Arg |
| | | | | | | | (37%); FLT3 |
| | | | | | | | p.Asp835Tyr |
| | | | | | | | (4%); FLT3 |
| | | | | | | | p.Asp835Pro |
| | | | | | | | (2%); VUS in |
| | | | | | | | WT1 |
| | | | | | | | p.Arg158His |
| | | | | | | | (50%) |
| APL | 1+ | Yes, MK | Yes, MK, | 70 | No | 46, XX, t(15; 17)(q24; q21)[17]/46, XX[3] | N/A |
| | | | rare | | | | |
| | | | macrophages | | | | |
| APL | 1+ | Yes, MK | Yes, MK, | 80 | No | 46, XY, t(15; 17)(q24; q21)[11]/46, XY[9] | N/A |
| | | | macrophages | | | | |
| APL | 2+ | Yes, MK | Yes, MK | 70 | No | 46, XY, t(15; 17)(q24; q21)[15]/46, XY[5] | N/A |
| Complex | 2+ | Yes, MK | Yes, MK | 90 | Yes | 56~57, XY, +Y, +6, +8, +8, t(9; 11)(p22; | U2AF1 |
| karyotype | | | | | | q23), +11, | p.Ser34Phe |
| | | | | | | add(12)(p11.2), +14, +18, +18, +19, +21, +22[cp19]/ | (6%) |
| | | | | | | 46, XY[1] | |
| Complex | 3+ | Yes, MK, | Yes, MK, | 30 | No | 45, XY, +1, +add(1)(p13), psu | TP53 p.Arg342* |
| karyotype | | ?myeloids | macrophages? | | | dic(1; 1)(q21; q21), −2, add(2)(p11.2), −7, | (57%); BCOR |
| | | | | | | del(9)(q13q22), der(12)del(12)(p13) | p.Ser423Phefs* |
| | | | | | | add(12)(q13), add(13)(q22), | 16 (25%); VUS |
| | | | | | | add(17)(p11.2), −20, +mar[cp7]/46, XY[13] | in BCORL1 |
| | | | | | | | (6%) |
| Complex | 2+ | Yes, rare | Yes, MK, | 70 | No | 45, XY, ?inv(17)(p13q21), −20, | BCOR |
| karyotype | | MK | macrophages | | | del(21)(q22)[11]/45, XY, del(6)(p21), | p.Gln1208Thrfs |
| | | | | | | add(11)(p15), −17, | *8 (89%); TP53 |
| | | | | | | del(21)(q22)[7]/45, XY, del(4)(q25), −17[2] | p.Arg306* |
| | | | | | | | (86%) |
| Monosomal | 3+ | Y | Yes, rare | 50 | No | 45, XX, add(1)(q21), −5, | TP53 |
| | | | cells | | | add(6)(q25), +8, −13, −15, −17, −17, | p.Gly244Asp |
| | | | | | | ?del(21)(q22), +3mar[6]/46, XX[14] | (52%) |
| Monosomal | 1+ | Y, MK, | Yes, rare | 25 | No | 43~44, XX, del(3)(q21), add(7)(p21), | TP53 |
| | | myeloids | plasma cells | | | ?i(9)(q10), −9, −9, −18, add(18)(q23), | p.His 179Arg |
| | | | | | | add(21)(p11.2)[cp18]/46, XX[2] | (25%) |
| Monosomal | 1+ | Y, MK, | Yes, MK | 60 | No | 43~47, XX, del(5)(q13q33), −7, +8, | TP53 |
| | | myeloids | | | | add(8)(q24), −10, −11, add(12)(p13), | p.Leu111Pro |
| | | | | | | add(13)(p11.2), −14, −16, −22, +r, +1~4mar[cp19]/ | (72%); VUS in |
| | | | | | | 46, XX[1] | ZRSR2 |
| | | | | | | | p.Ser447_Arg4 |
| | | | | | | | 48insGlnSer |
| | | | | | | | (46%) |
| AML | 1+ | Yes, MK, | Yes, PC | 90 | No | 46, XX[20] | GATA2 |
| | | PC (rare | (rare cells) | | | | p.Ala318Thr |
| | | cells) | | | | | (13%) |
| AML | 0 | Yes, MK, | Yes, MK, | 90 | No | 47, XX, +13[16]/48, idem, +13[1]/46, | ASXL1 |
| | | rare | rare | | | XX[3] | p.Glu635Argfs* |
| | | myeloids | macrophages | | | | 15 (40%); |
| | | | | | | | BCOR |
| | | | | | | | p.Glu1076Glyfs |
| | | | | | | | *3 (37%); |
| | | | | | | | RUNX1 |
| | | | | | | | p.Ser388 |
| | | | | | | | (85%); SRSF2 |
| | | | | | | | p.Pro95His |
| | | | | | | | (48%) |
| AML | 3+ | Yes, MK | Yes, MK, | 90 | No | 46, XY, del(7)(q22)[14]/46, XY[6] | FLT3-ITD p.? |
| | | | rare | | | | (1%); FLT3-ITD |
| | | | macrophages | | | | p.Lys602_Trp60 |
| | | | | | | | 3ins6 (<1%); |
| | | | | | | | IDH1 |
| | | | | | | | p.Arg132His |
| | | | | | | | (5%); NRAS |
| | | | | | | | p.Gly12Asp |
| | | | | | | | (4%); PTPN11 |
| | | | | | | | p.Glu76Ala |
| | | | | | | | (35%); RUNX1 |
| | | | | | | | p.Gln415Profs* |
| | | | | | | | 185 (46%); |
| | | | | | | | U2AF1 |
| | | | | | | | p.Ser34Phe |
| | | | | | | | (44%); VUS in |
| | | | | | | | BCOR |
| | | | | | | | p.Arg1375Trp |
| | | | | | | | (91%) |
| Monocytic | 3+ | Yes, MK, | Yes, MK | 50 | Yes | 47, XX, +8[4]/46, XX[16] | DNMT3A p.? |
| AML | | ?myeloids | | | | | (45.2%); NPM1 |
| | | | | | | | p.Trp288Cysfs* |
| | | | | | | | 12 (34.7%); |
| | | | | | | | FLT3 |
| | | | | | | | p.Asp835Tyr |
| | | | | | | | (34.7%); FLT3- |
| | | | | | | | ITD |
| | | | | | | | p.Gly583_Tyr59 |
| | | | | | | | 9dup (1.3%); |
| | | | | | | | VUS in ASXL1 |
| | | | | | | | p.Lys1532* |
| | | | | | | | (VAF not |
| | | | | | | | available) |
| Monocytic | 3+ | No/NA | No/NA | 90 | Yes | 46, XX[20] | NPM1 |
| AML | | | | | | | p.Trp288Cysfs* |
| | | | | | | | 12 (27.7%); |
| | | | | | | | TET2 p.Gln321* |
| | | | | | | | (31.9%); TET2 |
| | | | | | | | p.Lys1493Serfs |
| | | | | | | | *78 (31.8%) |
| Monocytic | 3+ | Yes, MK | Yes, MK, | 90 | Yes | 46, XY[20] | FLT3 |
| AML | | | rare | | | | p.Asp835Tyr |
| | | | macrophages | | | | (41%); NPM1 |
| | | | | | | | p.Trp288Cysfs* |
| | | | | | | | 12 (47%) |

AML, acute myeloid leukemia;
APL, acute promyelocytic leukemia;
MK, megakaryocyte;
N/A, not applicable;
NGS, next generation sequencing;
PC, plasma cell;
VAF, variant allele frequency

TABLE 2
List of AML patients for flow cytometry

| | | | PD-1H |
|---|---|---|---|
| Diagnosis | Phenotype | Cytogenetics | delta MFI |
| ACUTE MYELOID | CD14+ CD64+ CD11b+ | 49, XY, +4, +8, +21[3]/46, XY[12] | 12374 |
| LEUKEMIA WITH | CD33+ CD13variable+ | | |
| MYELODYSPLASIA- | CD16subset+ CD34− | | |
| RELATED | CD117− with aberrant CD56 | | |
| CHANGES/Monocytic | positivity and HLADr | | |
| Feature | negativity, most consistent | | |
| | with | | |
| | monoblasts/promonocytes. | | |
| ACUTE MYELOID | CD45dim+ CD13+ CD34+ | 46, XY, del(9)(p21)[3]/45, idem, −Y[12] | 1851 |
| LEUKEMIA | CD7dim− HLA-DR+ CD33+ | | |
| INVOLVING 30% OF | MPO+, CD117var+ | | |
| TOTAL | | | |
| CELLULARITY | | | |
| ACUTE MYELOID | CD34+ CD117+ | 47, XX, del(5)(q13q34), +8[17] (del5 q and tri 8 in a subclone) | 424 |
| LUKEMIA | HLADRdim+ CD13+ | | |
| INVOLVING 20% OF | CD33var+ CD11b− CD7− | | |
| BONE MARROW | CD64− CD14− CD10− | | |
| CELLULARITY | | | |
| PERSISTENT | CD45dim+ CD13dim+ | XY[20] | 695 |
| MYELOID | CD34+ CD117+ CD33+ | | |
| NEOPLASM WITH | HLADR+ MPO+ | | |
| 13% BLASTS | myeloblasts with very dim, | | |
| (Erythroid dysplasia) | aberrant CD7 expression. | | |
| ACUTE MYELOID | CD45dim+ CD13+ CD34+ | 46, XY, del(9)(q21q32)[5]/46, XY[10] | 675 |
| LEUKEMIA, 40% | CD117+ CD33+ HLADR+ | | |
| BLAST | CD38+ MPO+ TdT−. Blasts | | |
| | also demonstrate dim, | | |
| | aberrant CD7 expression. | | |
| ACUTE MYELOID | CD45dim+ CD34dim+ TdT− | 46, XX[20] | 1259 |
| LEUKEMIA (90% | MPO++ CD117dim+ CD13+ | | |
| BLASTS) | CD33+ HLADRdim+ CD14− | | |
| | CD64− CD56− CD7− | | |
| CMML-->AMML | CD38++ CD15− CD56− | 46, XX, del(4)(q21.23q25)[7]46, idem,r(10)(p11.2q23)[3]/46, idem, | 15320 |
| | CD19− CD2− CD7−. (Marked | der(10)t(3; 10)(q21; p11.2)[3]/ /47, idem, +8, | |
| | increased monocytes with | der(13)t(3; 13)(q21; p11.2)[cp2]/46, idem, | |
| | approximately 28-30% of | der(22)t(3; 22)(q21; q13)[1] | |
| | total marrow cellularity | | |
| | consists of CD64+ | | |
| | monocytes with variable | | |
| | CD14 expression and dim | | |
| | expression of CD56. There | | |
| | are normal numbers of | | |
| | myeloblasts with normal | | |
| | myeloid scatter by | | |
| | CD45/SSC. There is an | | |
| | abnormal | | |
| | CD10/CD13/CD16/CD11b | | |
| | myeloid maturation pattern | | |
| | suggesting myeloid | | |
| | dysmaturation) | | |
| ACUTE MYELOID | CD34+ CD117subset+ | 46, XY, t(9; 22)(q34; q11.2)[15] | 963 |
| LEUKEMIA | CD33− CD7− CD13+ | | |
| INVOLVING 30-40% | CD11b− CD16− CD2− CD10− | | |
| OF TOTAL BONE | CD19− CD5− HLADR+ | | |
| MARROW | CD64− glycophorin− CD41a− | | |
| CELLULARITY | TDT var+ CD79a−, MPO+ | | |
| | CD45dim+ | | |
| Acute monocytic | CD34+ CD117− CD33+ HLA | 46, XY[20] | 25000 |
| leukemia (AML M5) | DR+ MPO− CD13+ (subset) | | |
| | CD11B+ CD64+ | | |
| Acute | CD45dim+ CD34− CD13+ | 46, XX[18] | 3122 |
| myelomonocytic | CD33+ HLADR+ CD117+ | | |
| leukemia (AML M4) | CD7− CD11b dim/− CD16− | | |
| | CD64dim+. | | |
| Acute | About 15-17% of total | 45, Y, der(X)t(X; 7)(q22; q22), t(1; 12)(q31; q21), | 2840 |
| myelomonocytic | circulating cells are | del(3)(p21p23), add(3)(q21), der(4)t(4; 15)(q21; q15), | |
| leukemia (AML M4) | CD45dim+ CD13+ CD34+ | del(5)(q15), −7, del(15)(q15)[cp14]/46, XY[1] | |
| | CD117+ CD33dim+ HLA− | | |
| | DR+ MPO− myeloblasts. In | | |
| | addition, about 30% of total | | |
| | cells are monocytes by | | |
| | immunophenotype | | |
| | (CD45dim+ CD13+ | | |
| | CD11bdim+ HLA-DRdim+ | | |
| | CD33+ CD14dim+ | | |
| | CD16dim+ CD64+ | | |
| | CD4dim+ CD34− CD117− | | |
| | MPO−) | | |
| Acute | CD33+ CD13dim+ | 48, XX, +6, t(11; 17)(q23; q25), +13[15]/50, idem, +4, +8[3]/46, XX[2] | 243 |
| myelomonocytic | CD64dim+ CD56− CD38+ | (KMT2A gene rearrangement) | |
| leukemia (AML M4) | CD14− MPOsubset+ | | |
| | CD117− CD34− HLADR+ | | |
| | CD11bdim MPO subset+ | | |
| AML | CD45dim+ CD34dim/− | XX[20] | 601 |
| | CD33+ CD117var+ CD7− | | |
| | HLADRvar+ CD56− CD14− | | |
| | CD64dim+ CD38var+ | | |
| | MPO+. | | |
| AML with monocytic | CD117(subset)+ CD34− | 46, XX[20] | 21103 |
| differentiation | CD33+ HLADR− CD13+ | | |
| | CD14− CD4− CD64− CD11b− | | |
| | CD19dim+ CD10− TdT− | | |
| | CD79A− MPO(variable) | | |
| | CD3− CD45dim+ | | |
| Poorly differentiated | CD45dim+ CD13+ CD34+ | 46, XX, del(1)(p13p31)[6]/46, XX[9] | 260 |
| acute myeloid | CD117+ HLADR+ | | |
| leukemia | CD33dim+ CD11b− CD16− | | |
| | CD19− CD10− CD7− CD56− | | |
| | CD3− CD64− MPOdim/− | | |
| | TdT− CD79a− | | |
| AML with monocytic | CD34+ CD117+ TdT− | 46, XY, del(21)(q21q22)[9]/46, XY[12] (21q deletion does not | 917 |
| differentiation | CD45− CD13+ MPO(var)+ | include the RUNX1 gene at 21q22.12) | |
| | CD7(dim)+ CD19− CD79a− | | |
| | CD5− CD8− CD10− CD16− | | |
| | CD56− CD11b− cytoCD3− | | |
| | HLADR+ CD33(dim)+. A | | |
| | subset of cells are | | |
| | CD4(dim)+ CD14+ | | |
| | CD45dim)+ SSClow | | |
| Relapsed AML- | 70% of total cells with a | 46, XX, t(13; 16)(q22; p13.3)[20] | 10367 |
| myelomonocytic | CD45dim+, CD34+ | | |
| | CD117dim+, HLADRdim/−, | | |
| | CD7−, CD13+, CD19−, | | |
| | MPO+, TdT−, CD33+ | | |
| | myeloblast | | |
| | immunophenotype. | | |
| | Approximately 22% of cells | | |
| | are CD45dim+, CD13+, | | |
| | CD34−, CD117dim+, | | |
| | CD33+, HLADRdim/−, | | |
| | CD64dim+ blasts likely | | |
| | reflecting an immature | | |
| | monocyte phenotype. | | |
| AML with monocytic | 64% of total cells are | 50, XX, +i(5)(p10)x3, +8[8]/46, XX[7] | 25088 |
| differentiation | variable side scatter, | | |
| | CD45dim+ CD34− CD117− | | |
| | CD33+ CD64dim+ CD4+ | | |
| | CD14− CD56+ CD11bvar+ | | |
| | CD13− MPO++ CD16− | | |
| | Glyco− CD41a− (negative for | | |
| | all T- and B-cell markers) | | |
| | myeloblasts with some | | |
| | evidence of monocytic | | |
| | differentiation | | |
| AML t(8; 21) | low SSC/CD45dim+ blasts, | 46, XY, t(8; 21)(q21.3; q22)[15] | 944 |
| | which are CD34+ CD117+ | | |
| | HLADR+ CD33dim+ CD13+ | | |
| | CD7− CD19dim+ MPOdim+ | | |
| | CD11b− CD64dim+ and | | |
| | CD4subset[dim]+. The | | |
| | blasts do not label for any | | |
| | other B or T cell markers. | | |
| AML t(8; 21) | CD45dim+ CD34+ HLA− | 46, XX, t(8; 21)(q21.3; q22)[20] | 323 |
| | DR+ CD117+ CD33+ | | |
| | CD13+ MPO+ CD7− CD16− | | |
| | CD11b− glycophorin A− | | |
| | CD41a−, CD56subset+ | | |
| | CD64dim/− CD14− TdT− | | |
| AML t(15; 17) | CD13+ CD33++ CD117+ | 46, XY, t(15; 17)(q24.1; q21.2)[14]/46, | 1968 |
| | CD34− HLA-DR− CD64+ | XY[1] | |
| | CD11b− CD16−. | | |
| AML t(8; 21) | High SSC CD34+ | 46, XX, t(8; 8; 21)(q22; p21; q22)[10]/47, idem, +4[3]/46, XX[3] ish | 338 |
| | CD16/56+ CD117variable+ | t(8; 8; 21)(q22; p21; q22)(RUNX1T1+, | |
| | HLA-DR+ CD13variable+ | RUNX1+; RUNX1T1+; RUNX1+) | |
| | MPO+ CD33− CD11b− | | |
| | CD2dim− CD64− CD14− | | |
| | CD19− CD20− CD3− CD7− | | |
| | CD5− CD10− CD41a− | | |
| | glycophorin a− TdT− CD79a− | | |
| AML t(8; 21) | MPO++ CD34(variable)+ | 46, XX, t(8; 21)(q22; q22)[15] | 601 |
| | CD117(variable)+ TdT− | | |
| | CD79a− HLADR+ CD13+ | | |
| | CD33− CD2− CD16− CD10− | | |
| | CD19− CD56dim+ CD14− | | |
| | CD64− CD41a− Glyco− | | |
| AMML | CD45dim+ CD33+ CD14+ | 46, XY[21] | 28227 |
| | HLADR+ CD13+ CD4dim+ | | |
| | CD11b+ CD16− CD64+ | | |
| | CD56+ CD8− MPO+ with a | | |
| | small population of CD34+ | | |
| | CD117+ CD33+ HLADR+ | | |
| | myeloblasts (2.5%). | | |
| AML (inversion 16) | Approximately 25% of cells | 46, XY, inv(16)(p13.1q22)[15] Abnormal clone detected. | 491 |
| | are immature myeloid | | |
| | elements by CD13/SSc | | |
| | criteria. About 7-8% of cells | | |
| | are CD13+ CD34+ | | |
| | myeloblasts and the | | |
| | remaining cells are CD64+ | | |
| | CD14variable+ monocytes. | | |
| | There is also evidence of | | |
| | myeloid dysmaturation by | | |
| | CD10/CD13/CD16/CD11b | | |
| | pattern. | | |

AML, acute myeloid leukemia;
AMML, acute myelomonocytic leukemia;
CMML, chronic myelomonocytic leukemia;
MFI, mean fluorescence intensity

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a leukemia in a subject, comprising administering to the subject a therapeutically effective amount of a checkpoint inhibitor and a therapeutically effective amount of an antibody that specifically binds PD-1H.

2. The method of claim 2, wherein the leukemia comprises an acute myeloid leukemia (AML).

3. The method of claim 2, wherein the leukemia comprises a myelodysplastic syndrome (MDS).

4. The method of claim 2, wherein the leukemia comprises a Philadelphia chromosome-positive acute lymphoblastic leukemia (Ph+ ALL).

5. The method of claim 1, wherein the checkpoint inhibitor comprises an anti-PD-1 antibody, anti-PD-L1 antibody, anti-CTLA-4 antibody, or a combination thereof.
